(19) United States
(12) Patent Application Publication
(10) Pub. No.: US 2025/0134404 A1
(43) Pub. Date: May 1, 2025

(54) LASER SPECKLE IMAGING DEVICE AND METHOD

(71) Applicant: Covidien LP, Mansfield, MA (US)

(72) Inventors: Jason Chun-Yip Kwok, Anaheim, CA (US); Sean M. White, Rancho Santa Margarita, CA (US)

(21) Appl. No.: 18/926,483

(22) Filed: Oct. 25, 2024

Related U.S. Application Data
(60) Provisional application No. 63/593,859, filed on Oct. 27, 2023.

(51) Int. Cl. A61B 1/00; A61B 1/06; A61B 1/227; A61B 1/233; G02B 27/48

(52) U.S. Cl. CPC A61B 5/0261; A61B 5/0042; A61B 5/6817; A61B 5/6819; A61B 2562/0238

(57) ABSTRACT

A system include a light source configured to emit quasi-coherent light to tissue of a patient, the light source configured to emit the quasi-coherent light through at least one of a nasal passage or an ear canal of the patient. The system includes a detector configured to detect resulting light passing through at least a portion of the tissue and generate an interference pattern image. The interference pattern image is representative of the portion of the tissue interacting with the quasi-coherent light traveling through the nasal passage or the ear canal of the patient. The system includes processing circuitry configured to receive the interference pattern image from the detector, generate a speckle contrast signal based on the interference pattern image, determine, based on the speckle contrast signal or a metric derived therefrom, a physiological characteristic of the patient, and output a representation of the physiological characteristic of the patient.

### CROSS-REFERENCE TO RELATED APPLICATIONS

This application claims the benefit of and priority to U.S. Provisional Patent Application Ser. No. 63/593,859 filed Oct. 27, 2023, the entire disclosure of which is incorporated by reference herein.

### TECHNICAL FIELD

This disclosure relates to blood flow monitoring.

### BACKGROUND

Various technologies can be used to monitor aspects of blood flow. For example, photoplethysmography (PPG) is an optical technique for assessing blood volume changes in the arteries during the cardiac cycle. Following each systolic and diastolic phase of the heart, the arteries are thought to undergo subtle volumetric expansion and contraction, respectively, which changes the light absorption signal measured by PPG. In this way, PPG can provide a “waveform” for the cardiac cycle, which can be used to assess vitals of a subject, such as heart rate and oxygen saturation. As another example, Laser Speckle Imaging (LSI) is an optical technology for measuring blood flow.

### SUMMARY

This disclosure describes devices, systems, and techniques for determining a physiological characteristic of a patient, such as blood flow, the occurrence of a stroke, e.g., ischemic stroke and/or hemorrhagic stroke, using Laser Speckle Imaging (LSI). A system can generate a laser speckle imaging signal that is representative of blood flow within a tissue region of a patient, such a brain of the patient. The laser speckle imaging signal can change with pulsatile flow during a cardiac cycle of the patient, leading to a blood flow waveform over time. In some examples, the system is configured to analyze separate components of one or more waveforms of the laser speckle imaging signal, such as flow value and waveform shape, to determine a blood flow metric representative of the blood flow state of the tissue and/or other physiological characteristics of the patient, e.g., stroke. The system can output the physiological characteristic, blood flow metric, and/or a related diagnostic metric for use by another device and/or for display to a user.

In one example, a system includes: a light source configured to emit at least quasi-coherent light to tissue of a patient, the light source configured to emit the at least quasi-coherent light through at least one of a nasal passage or an ear canal of the patient; a detector configured to detect resulting light passing through at least a portion of the tissue and generate an interference pattern image, wherein the interference pattern image is representative of the portion of the tissue interacting with the quasi-coherent light traveling through (e.g., emitted through) at least the nasal passage or the ear canal of the patient, and processing circuitry configured to: receive the interference pattern image from the detector; generate a speckle contrast signal based on the interference pattern image; determine, based on the speckle contrast signal or a metric derived from the speckle contrast signal, a physiological characteristic of the patient; and output a representation of the physiological characteristic of the patient.

In another example, a method includes: positioning a light source to emit at least quasi-coherent light to tissue of a patient through at least one of a nasal passage or an ear canal of the patient; positioning a detector to detect resulting light passing through at least a portion of the tissue; generating, by the detector and based on the resulting light, an interference pattern image, wherein the interference pattern image is representative of the portion of the tissue interacting with the quasi-coherent light traveling through at least the nasal passage or the ear canal of the patient; receiving, by processing circuitry, the interference pattern image from the detector; generating, by the processing circuitry, a speckle contrast signal based on the interference pattern image; determining, by the processing circuitry and based on the speckle contrast signal or a metric derived from the speckle contrast signal, a physiological characteristic of the patient; and outputting a representation of the physiological characteristic of the patient.

In another example, a laser speckle imaging system includes: a laser configured to emit at least quasi-coherent light to tissue of a patient, the laser configured to emit the at least quasi-coherent light through a nasal passage of the patient; a detector configured to: detect resulting light passing through at least a portion of the tissue and through an ear canal of the patient; and generate a laser interference pattern image of the tissue, wherein the laser interference pattern image is representative of the portion of the tissue interacting with the at least quasi-coherent light traveling through the nasal passage of the patient; and processing circuitry configured to: receive the laser interference pattern image from the detector; generate a speckle contrast signal or a metric derived from the speckle contrast signal based on the laser interference pattern image; determine, based on the speckle contrast signal or the metric derived from the speckle contrast signal, an occurrence of a stroke; and output a representation of the occurrence of the stroke.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual block diagram illustrating an example detection device.

FIG. 2 is a conceptual block diagram illustrating an example detection device configured to monitor a blood flow state of at least a portion of a patient.

FIG. 3 is a conceptual diagram illustrating an example positioning of a laser light emitting source and a detector of a laser speckle imaging system relative to anatomy of a patient and relative to a physiological characteristic of the patient.

FIG. 4 is a conceptual diagram illustrating another example positioning of a laser light emitting source and a detector of a laser speckle imaging system relative to anatomy of a patient and relative to a physiological characteristic of the patient.

FIG. 5 is a conceptual diagram illustrating another example positioning of a laser light emitting source and a detector of a laser speckle imaging system relative to anatomy of a patient.

FIG. 6 is a conceptual diagram illustrating another example positioning of a laser light emitting source and a detector of a laser speckle imaging system relative to anatomy of a patient and relative to a physiological characteristic of the patient.

FIG. 7 is a conceptual diagram illustrating another example positioning of a laser light emitting source and a detector of a laser speckle imaging system relative to anatomy of a patient and relative to a physiological characteristic of the patient.

FIG. 8 is a conceptual diagram illustrating another example positioning of a laser light emitting source and a detector of a laser speckle imaging system relative to anatomy of a patient and relative to a physiological characteristic of the patient.

FIG. 9 is a conceptual diagram illustrating another example positioning of a laser light emitting source and a detector of a laser speckle imaging system relative to anatomy of a patient and relative to a physiological characteristic of the patient.

FIG. 10 is a conceptual diagram illustrating another example positioning of a laser light emitting source and a detector of a laser speckle imaging system relative to anatomy of a patient and relative to a physiological characteristic of the patient.

FIG. 11 is a conceptual diagram illustrating another example positioning of a laser light emitting source and a detector of a laser speckle imaging system relative to anatomy of a patient and relative to a physiological characteristic of the patient.

FIG. 12 is a flow diagram illustrating example techniques for determining a physiological characteristic of a patient using a laser speckle imaging system.

### DETAILED DESCRIPTION

This disclosure describes devices and techniques, such as Laser Speckle Imaging (LSI), that may replace other techniques such as computed tomography (CT) to detect blood flow in the vasculature and distinguish between high and low flow locations that may be indicative of a blood clot or hemorrhage or inadequate vessel closure, for example. CT is one example imaging modality, but it may have several disadvantages such as the use of ionizing radiation in the patient (which may cause cancer), the use of contrast to which a patient may be allergic and which takes time to travel through vasculature causing a delay in when a surgeon can check blood flow (and which needs to clear and dissipate from tissue before taking a new measurement), and common CT equipment are expensive and not portable (e.g., for use by paramedics).

LSI may also be used to replace other techniques such as CT and magnetic resonance imaging (MRI). MRI is used to inspect tissue for changes that may be indicative of an infarction or hemorrhage, but it may have several disadvantages such as its incompatibility with patients who have metallic implants (e.g., a pacemaker), its high cost, and its inability to be transported for use by paramedics.

LSI can include delivering coherent, or at least quasi-coherent, light, typically from a laser, to an area or volume of tissue or a tissue region of a patient, such a brain, a blood vessel, a digit (e.g., a finger or toe) or limb of the patient. The laser may be configured with optimal frequency and intensity for adequate tissue penetration. The tissue/material in the area of interest can cause the photons to interfere with one another and create a speckle pattern detected from light scattering within at least a portion of the tissue that received the coherent light. Tissue and/or material that is in motion (e.g., blood flowing through a blood vessel) creates a speckle pattern or part of a speckle pattern which changes at a different rate than the speckle pattern from slower moving or relatively stationary tissue and/or material (e.g., blood flowing more slowly, bone, connective tissue, fat). LSI software may be designed to interpret the speckle pattern and recreate an image of the vasculature, similar to a fluoroscopic image (but without ionizing radiation, dyes, and using different camera systems). LSI software may also be designed to interpret the speckle pattern and provide information on the blood flow rate.

The volume and tissue and location of interrogated tissue varies depending on the placement of the light source or sources (e.g., “laser light emitting source”) and the detector or detectors. According to the systems and techniques disclosed herein, an LSI system may be configured to be placed near a region of interest in a patient, e.g., measurement geometries could include using the laser light emitting source and detector which are spatially separated and placed on the scalp. Alternatively, the laser light emitting source may be placed within an oral or nasal passage (or nasal cavity) or an ear canal, with the light detector placed on the scalp, or in a different nasal passage or ear canal. In some examples, the light source and the light detector may be placed in the same type of orifice or exact same orifice, such as the nasal passage (or adjacent nasal cavities) or the same ear canal (or opposing ear canals).

The devices, systems, and techniques of this disclosure may include an LSI device comprising a laser light emitting source and one or more detectors configured to receive laser light from the laser light emitting source and separated from the laser light emitting source. For example, the one or more detectors and laser light emitting source may be housed in separate housings configured to be positioned at different positions or locations of the patient. For example, the one or more detectors and laser light emitting source may be housed in separate nasoscopes, otoscopes, or at different positions of a head mounted device, or any combinations thereof. The LSI device may then capture a laser speckle pattern of laser light having propagated through a volume of tissue, and processing circuitry may analyze the captured laser speckle pattern. The processing circuitry may then determine a physiological characteristic of the patient, such as a blood flow of the tissue through which the laser light propagated, and output a representation of the physiological characteristic of the patient.

The devices, systems, and techniques of this disclosure may present advantages over other systems. For example, devices, systems, and techniques of this disclosure may determine a physiological characteristic of a patient without the use of ionizing radiation, without the use of contrast and dyes, with lower cost equipment, with portable equipment, and with a faster response time, e.g., in real-time or near real-time.

In some examples, the devices, systems, and techniques of this disclosure may provide depth information regarding tissue of interest, via LSI imaging. For example, rather than a surface image of tissue, or an image close to the surface of tissue, the devices and techniques may provide information at a plurality of depths, e.g., the emitted light may propagate along an optical path through tissue to an appreciable depth within the tissue, or all the way through the tissue, and interact with the tissue along the optical path length (e.g., interactions including transmission, reflection, and scattering by the tissue). This deeper tissue may include blood vessels or other tissues that may be altered or change, indicative of a disorder or condition of the patient. In some examples, the devices, systems, and techniques of this disclosure may avoid including some portions of tissue of the patient along an optical path including target tissue, e.g., via LSI imaging through a nasal passage or ear canal to avoid requiring light to propagate through the full thickness of the head when desiring to measure blood flow in the brain by placing the emitter (light source) and detector or detectors as close as possible to target tissue. The example devices, systems, and techniques may provide improved or increased imaging or diagnostic accuracy, usability or speed of use, signal and/or signal to noise ratio, and may use less energy.

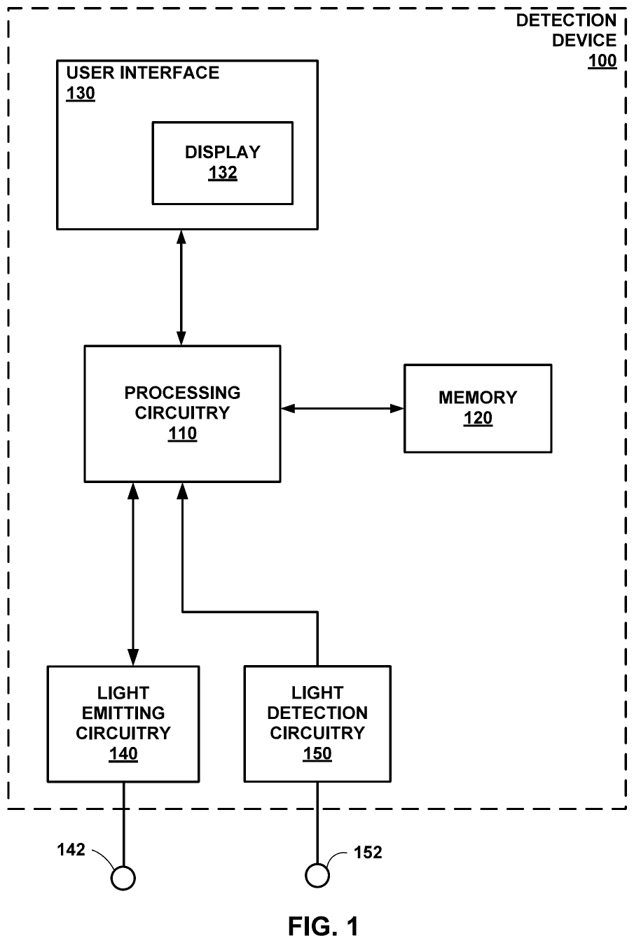

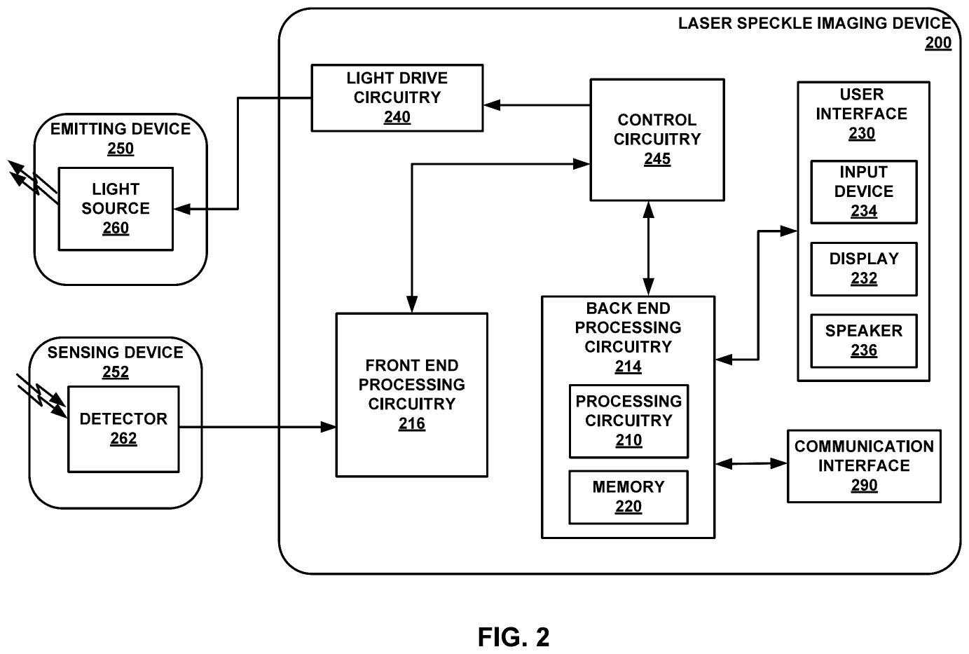

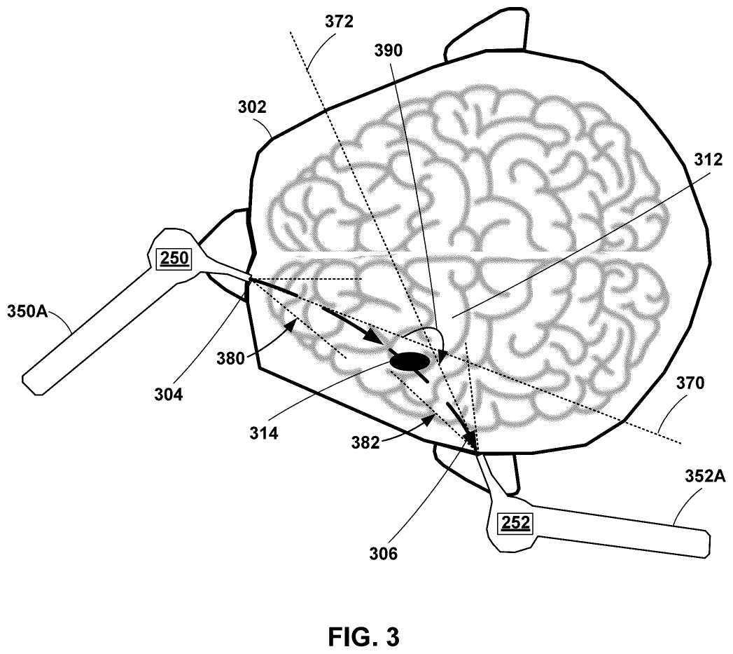

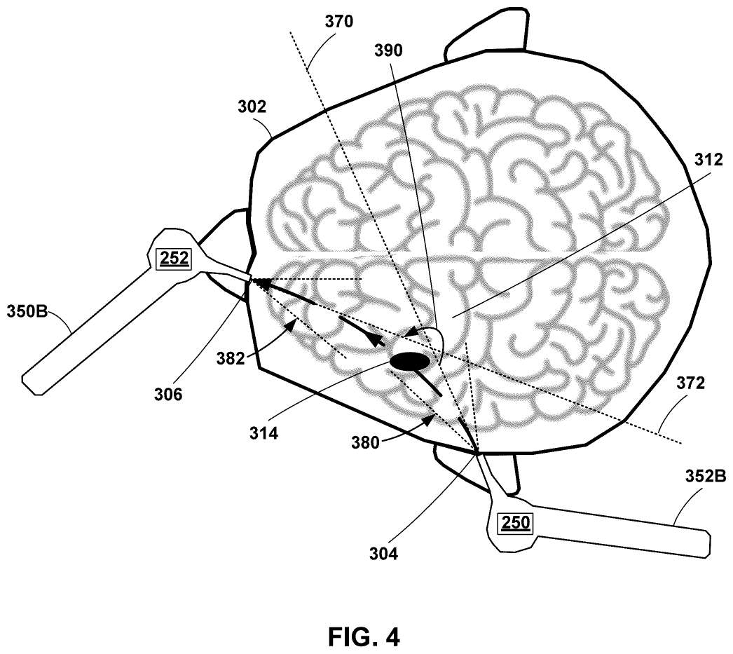

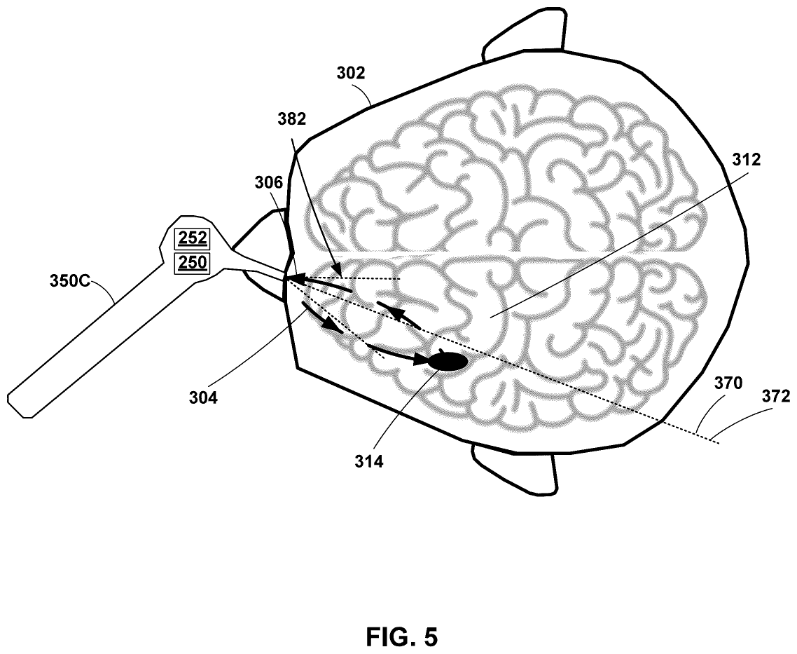

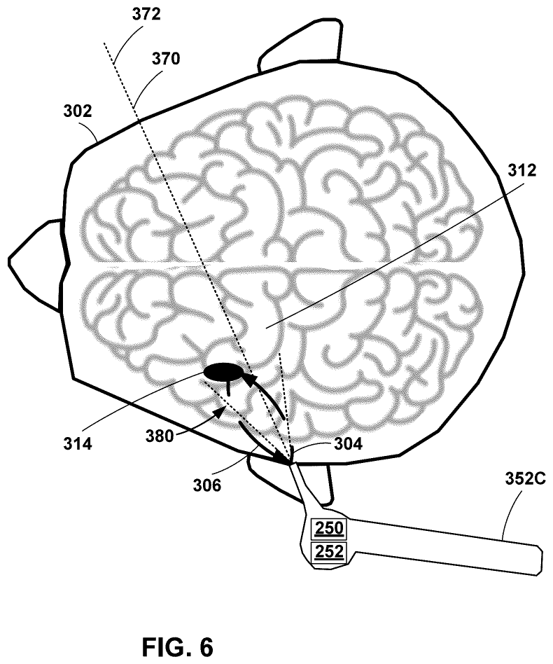

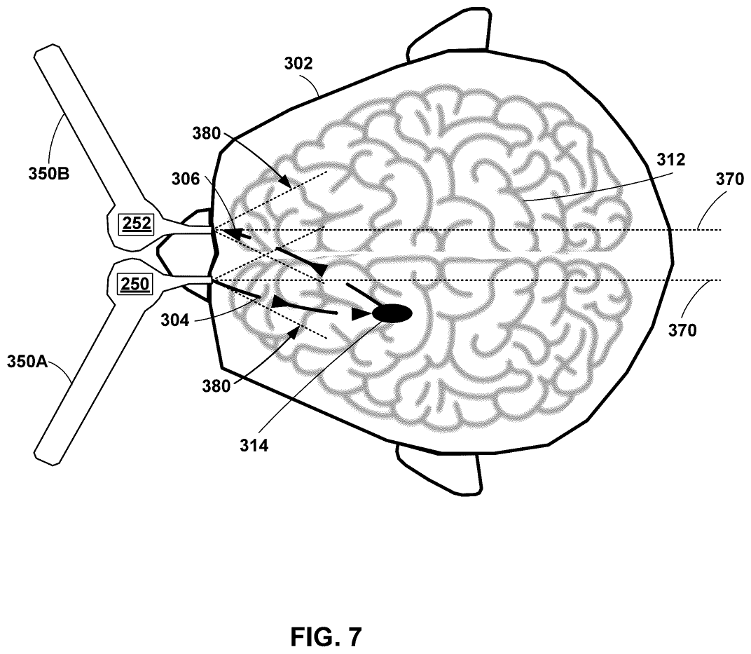

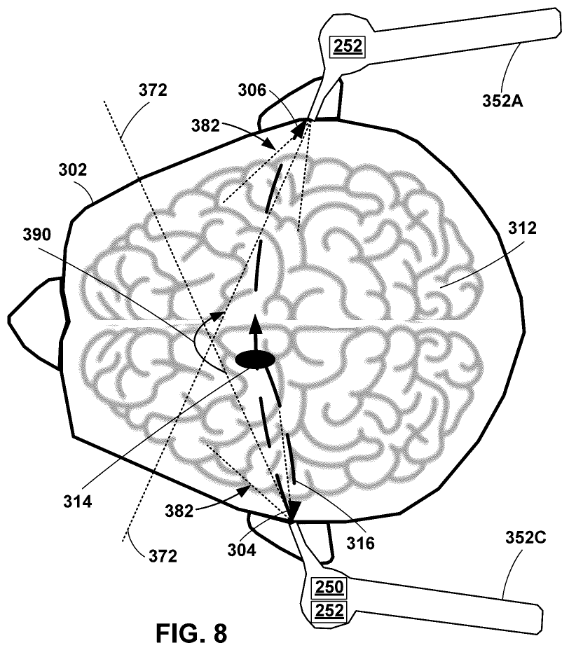

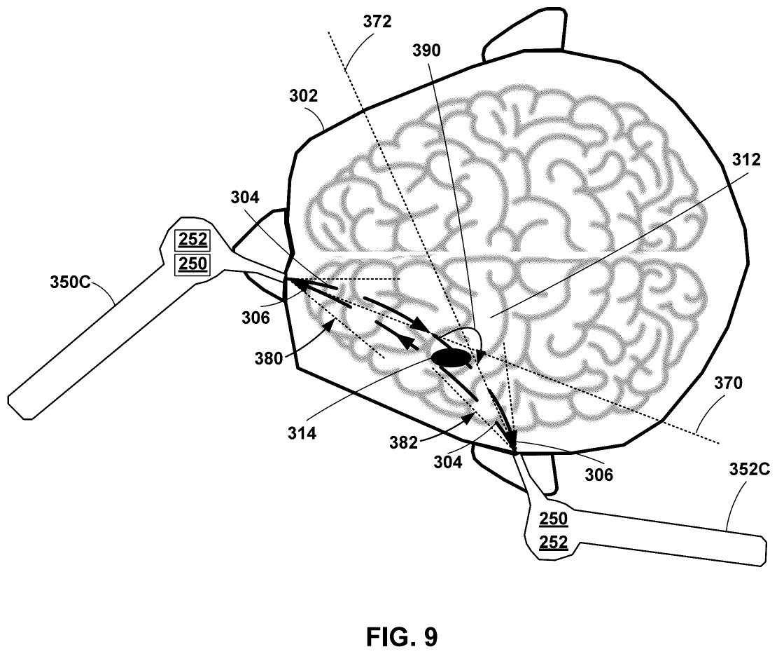

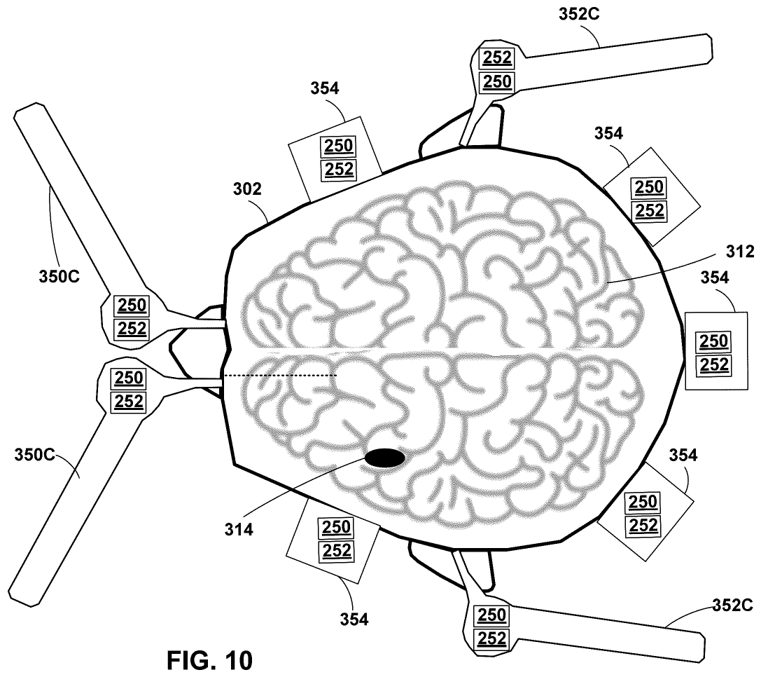

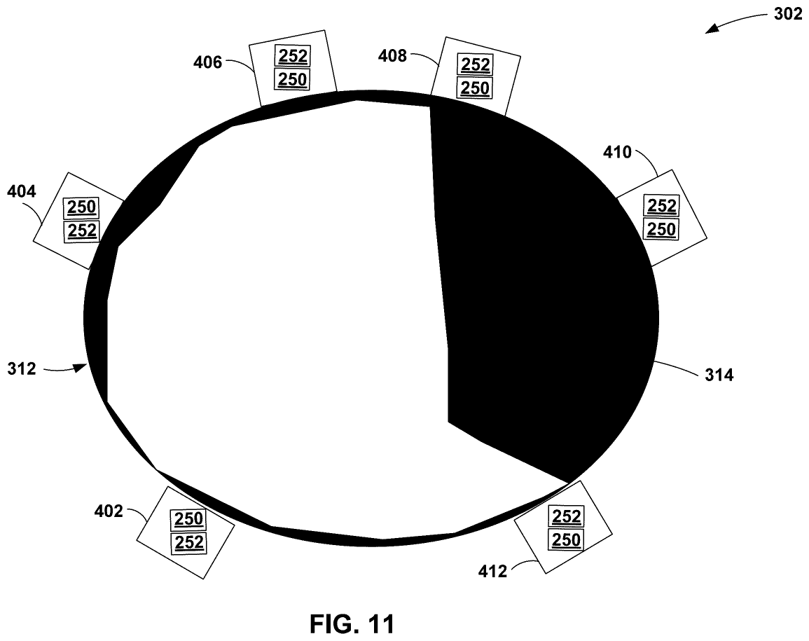

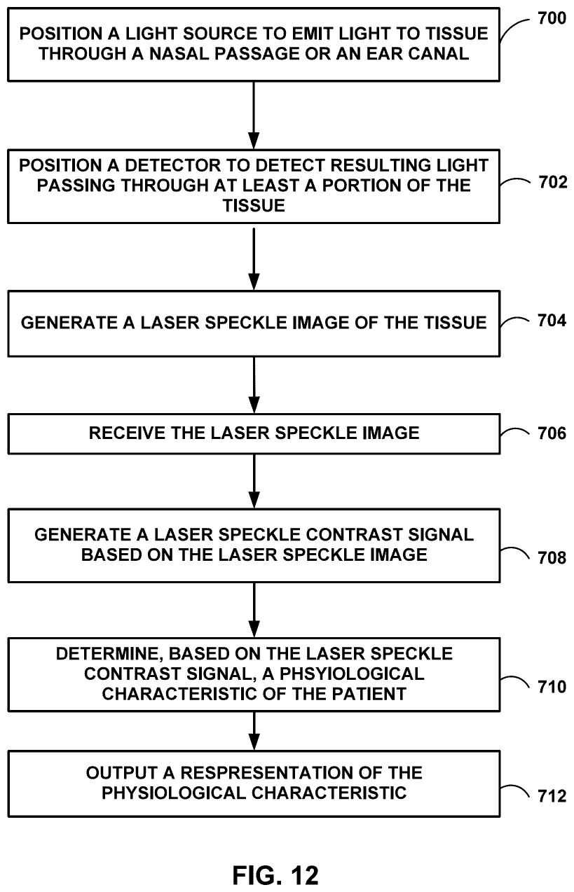

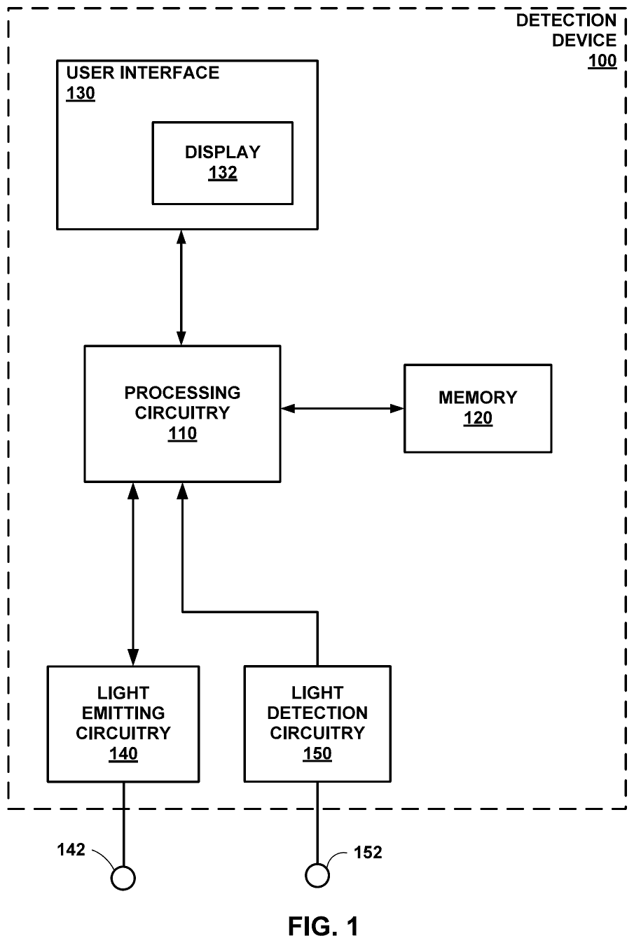

FIG. 1 is a conceptual block diagram illustrating an example detection device 100. The detection device 100 includes processing circuitry 110, a memory 120, a user interface 130 including a display 132, light emitting circuitry 140, a light source 142, light detection circuitry 150, and a light sensor 152. In some examples, the detection device 100 is configured to determine and display a physiological characteristic of a patient, e.g., a blood flow metric of a patient or tissue of a patient for diagnosis, during a medical procedure or for more long-term monitoring. A clinician may receive information regarding the physiological characteristic of a patient via the display 132 (or another output, such as audio circuitry configured to generate a sound) and diagnose or adjust treatment or therapy to the patient based on the physiological characteristic.

The processing circuitry 110 as well as other processors, processing circuitry, controllers, control circuitry, and the like, described herein, may include one or more processors. The processing circuitry 110 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, the processing circuitry 110 may include multiple components, such as any combination of one or more microprocessors, one or more DSPs, one or more ASICs, or one or more FPGAs, as well as other discrete or integrated logic circuitry, and/or analog circuitry.

The memory 120 may be configured to store data related to the physiological characteristic, such as raw signals from the light detection circuitry 150, LSI signals, or other information, for example. In some examples, the memory 120 may be configured to store information related to other sensed information from other sensors or devices, which may be displayed with the physiological characteristic in some examples. The memory 120 may also be configured to store information, such as instructions for determining the physiological characteristics, diagnostic metrics, characteristics of the LSI signal (e.g., flow and waveform metrics), controlling the light emitting circuitry 140 and the light detection circuitry 150, the controlling the user interface 130, or any other such information related to the operation of the detection device 100.

In some examples, the memory 120 stores program instructions, which may include one or more program modules, which are executable by the processing circuitry 110. When executed by the processing circuitry 110, such program instructions cause the processing circuitry 110 to provide the functionality ascribed to it herein. The program instructions may be embodied in software, firmware, and/or RAMware. The memory 120 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

The user interface 130 and/or the display 132 may be configured to present information to a user (e.g., a clinician). For example, the user interface 130 and/or the display 132 may be configured to present a graphical user interface to a user, where each graphical user interface may include indications of values of one or more physiological characteristics and/or physiological parameters of a subject. For example, the processing circuitry 110 may be configured to present blood pressure values, blood flow characteristics, physiological characteristic and/or parameter values, and indications of the physiological characteristic over time, or disease status of a patient via the display 132. In some examples, if the processing circuitry 110 determines that the physiological characteristic of the patient is indicative of an impaired condition, e.g., impaired blood flow indicative of a stroke or the like, then the processing circuitry 110 may present a notification (e.g., an alert) indicating the impaired condition of the patient status via the display 132.

The user interface 130 and/or the display 132 may include a monitor, cathode ray tube display, a flat panel display such as a liquid crystal (LCD) display, a plasma display, or a light emitting diode (LED) display, personal digital assistant, mobile phone, tablet computer, laptop computer, any other suitable display device, or any combination thereof. The user interface 130 may also include means for projecting audio to a user, such as audio generation circuitry and speaker(s). The processing circuitry 110 may be configured to present, via the user interface 130, a visual, audible, or somatosensory notification (e.g., an alarm signal) indicative of the patient's physiological characteristic. The user interface 130 may include or be part of any suitable device for conveying such information, including a computer workstation, a server, a desktop, a notebook, a laptop, a handheld computer, a mobile device, or the like. In some examples, the processing circuitry 110 and the user interface 130 may be part of the same device or supported within one housing (e.g., a computer or monitor). In other examples, a user interface separate from the detection device 100 can be configured to present information regarding physiological characteristics to a user, where such information may be provided to the user interface via the processing circuitry 110.

The light emitting circuitry 140 is configured to control light source 142 to generate light. For example, the light source 142 may include a coherent light source (e.g., a laser light source) emitting light having substantially the same frequency. Light sensor 152 (or light detector 152) includes one or more light sensitive structures configured to convert light energy into electric signals (e.g., a photoelectric device such as a charge-coupled device (CCD or some other sensor or detector). The light detection circuitry 150 may power and receive the electrical signals from the light sensor 152. The light detection circuitry 150 may then generate a raw signal representative of the light detected by the light sensor 152. In some examples, the light detection circuitry 150 may perform initial processing and/or analog to digital conversion of the electrical signals from the light sensor 152 to generate the raw signal that is usable by processing circuitry 110 to generate the LSI signal. In other examples, light detection circuitry 150 may include processing circuitry configured to generate the LSI signal. In some examples, light emitting circuitry 140 and/or light detection circuitry 150 control and process light emission and light detection using light source 142 and light detector 152, respectively. In other examples, one or both of light emitting circuitry 140 and light detection circuitry 150 may be housed outside of the housing of detection device 100. In this case, detection device 100 may still include interface circuitry that provides power and/or control signals to light emitting circuitry housed separately and/or receives signals from light detection circuitry housed separately.

In operation, the light source 142 and the light sensor 152 are each placed at a location or locations on parts of a body of a patient such that the light sensor 152 can detect light scattered from the light emitted by the light source 142. For example, the light source 142 and the light sensor 152 may be positioned within a nasal passage or an ear canal of the patient. In some examples, the light source 142 and the light sensor 152 may be physically separate from each other and separately placed on the patient. For example, the light source 142 may be placed in a first nasal passage, and the light sensor 152 may be placed in the other nasal passage, or an ear canal, or at a scalp of the patient, and/or the light source 142 may be placed in a first ear canal, and the light sensor 152 may be placed in the other ear canal passage, or a nasal passage, or at a scalp of the patient, the light source 142 may be placed at a first position at a scalp of the patient, and the light sensor 152 may be placed at a second position at a scalp of the patient, in a nasal passage, or in an ear canal, or at a scalp of the patient. As another example, the light source 142 and the light sensor 152 are part of the same sensor or supported by a single sensor housing. For example, the light source 142 and the light sensor 152 may be part of an integrated sensor system configured to non-invasively measure blood flow of the tissue between the light source 142 and the light sensor 152.

While an example detection device 100 is shown in FIG. 1, the components illustrated in FIG. 1 are not intended to be limiting. Additional or alternative components and/or implementations may be used in other examples.

Processing circuitry 110 is configured to generate a LSI signal using the raw signal from light detection circuitry 150, where the LSI system provides laser speckle contrast measurements over time. In some examples, LSI is an imaging methodology used to create image-based representations of blood flow in tissues of interest. In other examples, “LSI” may be used to interrogate tissue through a depth of tissue, e.g., along an optical path through the tissue, and may be used to create representations of physiological characteristics (e.g., blood flow) of tissue along the optical path. In some examples, such representations may be image-based (e.g., a two-dimensional representation at a depth plane), three-dimensional image-based (e.g., a data cube or a stack of images at a plurality of depths) or non-image based (e.g., indicative of one or more physiological metrics such as blood flow along one or more optical paths of the coherent light propagating through tissue from the light source to the detector). In other words, as used herein “LSI” refers to laser-speckle contrast measurement methods that are broader than laser-speckle “imaging,” e.g., to include both imaging and non-imaging methods indicative of physiological characteristics of tissue. LSI illuminates a part of a patient's body, e.g., an organ, a brain, a finger or toe, using coherent light. The presence and movement of blood within the illuminated body part interacts with the light moving through the tissue. Thus, coherent laser light is scattered within samples of interest, e.g., vasculature. These scattering events lead to a difference in optical path length among photons traversing through the tissue, e.g., via differences in actual length or optical index of refraction. The result is a speckle pattern that is typically detected and/or imaged using the light sensor 152 (e.g., a light detector), such as a camera with a finite exposure time. If the scattering objects (such as blood cells) are in motion, then the speckle pattern fluctuates in time and blurs during the camera exposure, e.g., the moving blood cells reduce the coherence of the light and reduce the contrast of the speckle pattern. The amount of blurring is related to flow and quantified using a parameter called the speckle contrast.

Various features of alteration can be used to extract information about the presence and flow of blood. Such features can include, for example, changes in detected light intensity and contrast within the observed light pattern, both of which are correlated with the movement of red blood cells. Analysis of changes in intensity and contrast within the observed light pattern over time then provides dynamic and quantitative feedback about alterations in a patient's blood flow and tissue perfusion, from which informed inferences may be made with respect to the physical state of a patient.

In some examples, the locations of the coherent light source 142 and the light sensor 152 are coupled to the movement of the tissue sample. For example, in the case of delivering light and/or sensing light through a nasal cavity of the patient or an ear canal of the patient, the housing of the source and sensor may be configured to be in contact with the nose or ear to minimize movement artifacts. As such, patient movement causes the light sensor 152 and the coherent light source 142 to similarly move. Accordingly, the field of view of the tissue sample does not change upon movement of the tissue sample. The coupling of the light sensor, coherent light source, and tissue sample may be facilitated by shortening the distance between the light sensor 152 and coherent light source 142. The distance between the light sensor 152 and the coherent light source 142 may be shortened by reducing the field of view of the light sensor 152 and forgoing the formation of a focused image, thereby eliminating the need for light-shaping optics. In other examples, an unfocused image may be preferred to be received by the light sensor 152.

In some examples, the light source 142 is configured to emit coherent light via an optical fiber coupled with a laser source. In these examples, the optical fiber may emit a portion of the coherent light emitted by the coherent light source 142. The location of the coherent light source 142 can be coupled to patient movement by fixing the location of the coherent light source 142 and/or the optical fiber with respect to the tissue sample. As used herein, “light source” or “coherent light source” can include coherent light emitted via an optical fiber and/or the coherent light source itself.

In some examples, the device 100 does not require the formation of a focused image on the light sensor 152 of the light sensor. Thus, some examples of the light source 142 and/or the light sensor 152 may forgo lenses, thereby reducing the cost and size compared to other LSI systems. In addition, other LSI systems require a light sensor with image-forming optics, whereas some examples devices 100 described herein may use a cheaper light sensor without image-forming optics, such as a photodiode.

In some examples, the light sensor 152 may include an opaque sheet with one or more apertures near the light sensor 152. The opaque sheet may modify the numerical aperture of the light collection system and thus optimize the speckle size incident on the light sensor, obviating the potential issue of under-sampling traditionally associated with unfocused images and small speckle sizes. Additionally, in some examples, the processing circuitry 110 determines perfusion using values using all the pixels in an image, instead of small sliding windows. By doing so, artifacts from streaking images are reduced.

In some examples, the light source 142 and the light sensor 152 are used in a transmission geometry. In a transmission geometry, the light source 142 and the light sensor 152 are positioned on opposite sides of a tissue of interest, e.g., opposite ear canals of the patient. Thus, the light sensor is configured to receive transmitted light that travels through the entire thickness of the tissue. As mentioned, because blood is very highly forward scattering, the usage of transmitted light rather than backscattered light provides a higher signal to noise ratio than a reflection geometry. In addition, transmission geometry enables signal acquisition from all of the blood vessels or the majority of blood vessels within the tissue of interest.

Furthermore, transmission geometry enables the light source 142 to be placed in close proximity to the sample of interest. In some examples, the light source 142 contacts the surface of the sample of interest. In LSI, the light exits from the field of view of the tissue in order to capture an accurate image. Otherwise, the light may obstruct the region of tissue being imaged. However, because the light is transmitted through the sample in a transmission geometry in some examples, rather than reflected back, transmission geometry enables the light source 142 to be placed in close proximity to the sample (region of tissue being imaged). However, in some examples, light source 142 and the light sensor 152 may additionally or alternatively be used in a reflectance and/or backscattered geometry.

In some examples, the light source 142 may be chosen to maximize transmission of the light through the tissue of interest. For example, the light source 142 may be a laser having a wavelength ranging from 300 nm to 1100 nm. The light source 142 may further be chosen to maximize speckle contrast at the light sensor 152. The light source 142 may be a single-mode laser diode or a fiber coupled helium-neon laser according to some examples. In some examples, the light source 142 includes adjustable power output to provide an adequate signal at the light sensor 152. In some examples, the light sensor 152 may be a camera with or without image-forming optics, such as a charge-coupled device (CCD) camera or a complementary metal-oxide semiconductor (CMOS) camera. The light sensor 152 may also be a camera without image-forming optics, such as a photodiode.

In some examples, the device 100 can include one or more polarizers that are configured to convert light before the light reaches the light sensor 152 and after it illuminates tissue (e.g., interrogates tissue) of a patient. Placing the polarizer to convert light before the light enters the tissue would not be as effective, because light entering scattering tissue such as the digit becomes depolarized as it is scattered. An optical filter, e.g., a spectral or wavelength filter, may also be included in order to filter out light not coming from the light source 142, e.g., ambient light.

Processing circuitry 110 may determine the physiological characteristic, e.g., blood flow (perfusion), within the tissue sample using the speckle pattern detected by the light sensor 152. For example, the light sensor 152 generates electrical signals representative of light intensity and the light detection circuitry 150 generates a raw signal that represents captured frames associated with different light intensity values detected. The light intensity values captured by the light sensor 152 are indicative of coherent light that was scattered by red blood cells as it illuminated the tissue. The coherent light illuminating the tissue renders an image, which is captured by the light sensor 152. The light sensor 152 is configured to adequately sample the speckle pattern despite the unfocused image. In some examples, speckle sizes are increased by an opaque sheet with one or more apertures which alters the overall numerical aperture of the light sensor 152.

Based on the light intensity values associated with the unfocused image as provided by the raw signal, the processing circuitry 110 may compute speckle contrast spatially, temporally, or spatio-temporally (a hybrid of spatial and temporal). To compute speckle contrast spatially, the processing circuitry 110 may utilize a group of pixels at different spatial locations within the same frame. To compute speckle contrast temporally, the processing circuitry 110 may utilize pixels from the same spatial location across a sequence of frames captured at different times. The processing circuitry 110 may also compute speckle using a spatio-temporal method, which is a hybrid of the temporal and spatial methods. In any case, the processing circuitry 110 may calculate the speckle contrast.

In some examples, the processing circuitry 110 is configured to determine the speckle contrast by at least using the following equation:

$$K = σ / 〈 I 〉 , \qquad ( 1 )$$

where K is contrast, o is the standard deviation of a group of pixel values and <I> is the average of a group of pixel values.

Generally, acquisition of the LSI signal does not require a focused image (e.g., the light sensor 152 received an unfocused image). Therefore, the processing circuitry 110 may determine speckle contrast temporally from only one pixel location in some examples. Accordingly, the detection device 100 may use a photodiode as the light sensor 152 in some examples. Photodiodes are cheaper than cameras with image-forming optics. It is believed that in some cases, perfusion measurements acquired by utilizing only one pixel location may be comparable in accuracy to those acquired by utilizing multiple pixel locations. In addition, perfusion measurements acquired by utilizing pixels from an unfocused image are comparable in accuracy to those acquired by laser Doppler. In other examples, device 100 can utilize a focused image to generate the LSI signal and determine the blood flow metrics as described herein.

In some examples, in addition to or instead of the other techniques described herein, the processing circuitry 110 is configured to determine the speckle contrast using the standard deviation across the entire image generated by light sensor 152 and light detection circuitry 150. Doing so reduces or even eliminates the artifacts in the “streaking images” that may otherwise result from using an unfocused image. If the object being imaged is moved during the imaging, the out of focus speckle pattern may translate. These artifacts may propagate to the speckle contrast image, creating “streaking images.” In-focus speckles do not translate, and thus the speckle contrast image does not exhibit the streaking images. The streaking in unfocused images may be eliminated by calculating the standard deviation across the entire image. In addition, streaking is eliminated because of the random direction of motion of blood perfusion.

After calculating the speckle contrast value K, the processing circuitry 110 calculate a physiological characteristic, such as perfusion. Processing circuitry 110 may calculate perfusion as:

$$Perfusion ∝ 1 / {K}^{2} \qquad ( 2 )$$

Other factors may affect this computation, including camera exposure time, camera noise, optical absorption, and the presence of static scatterers.

Using equation (2), the processing circuitry 110 may determine a metric of perfusion based on the computed speckle contrast value. For example, the LSI signal may indicate the perfusion metric as it changes over time. Moreover, the processing circuitry 110 may analyze the LSI signal in order to determine several characteristics, such as a flow and waveform metric. The processing circuitry 110 then determines a blood flow metric from the flow and waveform metric, for example, which provides a more complete representation of how blood is being moved within the tissue of interest.

As described herein, the device 100 can include processing circuitry 110 configured to receive the laser speckle image from light sensor 152 and light detection circuitry 150, and generate a speckle contrast signal based on a received signal indicative of the detected light from the light detection circuitry 150, where the detected light scatted by tissue from a coherent light source (e.g., the light source 142). The processing circuitry 110 may be configured to determine, from the speckle contrast signal, a physiological characteristic or characteristics of the patient. The processing circuitry 110 may be configured to output a representation of the physiological characteristic(s), such as a representation that is presented by display 132 or another output mechanism, such as audio generating circuitry. In some examples, the physiological characteristic of the patient comprises at least one of a flow value of a fluid (e.g., a blood flow value), an image of a plurality of flow values within vasculature of the tissue of the patient, an occurrence of an ischemic stroke, or an occurrence of a hemorrhagic stroke.

The processing circuitry 110 can control the user interface 130 and/or display 132 to present the representation of the physiological characteristic. The physiological characteristic may be displayed as a single value, a graph over time, or any other graphical, numerical, or textual representation. User interface 130 may present these metrics in real-time or nearly in real-time (e.g., with less than one second delay), via display 132.

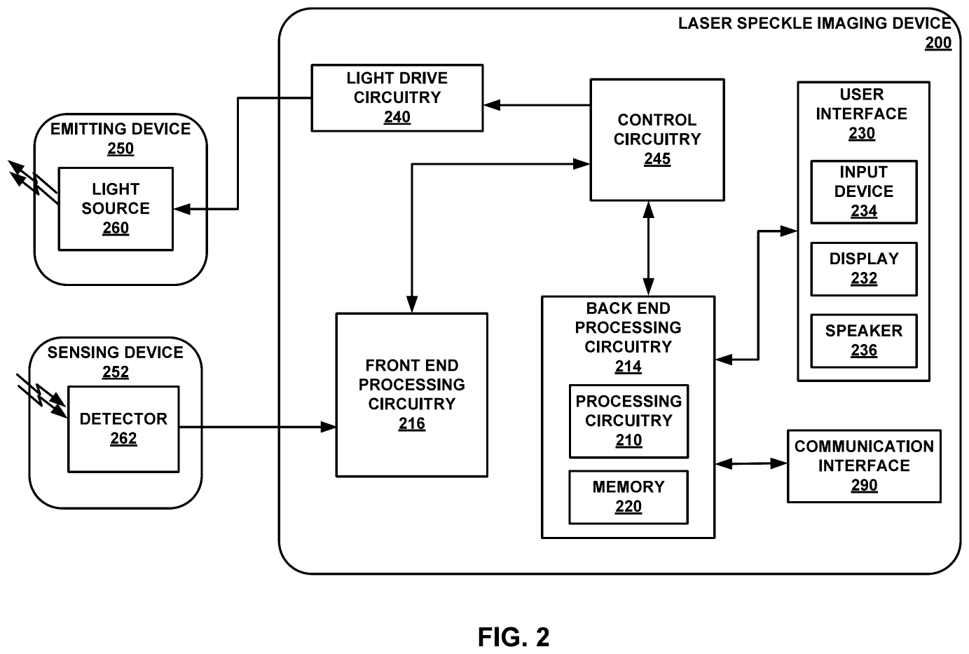

FIG. 2 is a conceptual block diagram illustrating an example laser speckle imaging (LSI) device 200 configured to monitor a physiological characteristic of at least a portion of a patient. In the example shown in FIG. 2, the LSI device 200 is coupled to an emitting device 250 and a sensing device 252 and may be collectively referred to as a physiological characteristic detection system, which generates and processes physiological signals of a subject and/or patient. In some examples, the emitting device 250, sensing device 252, and the LSI device 200 are part of a patient monitoring device. As shown in FIG. 2, the LSI device 200 includes the back-end processing circuitry 214, the user interface 230, the light drive circuitry 240, the front-end processing circuitry 216, the control circuitry 245, and the communication interface 290. The LSI device 200 is communicatively coupled to emitting device 250 and sensing device 252. The LSI device 200 is an example of the detection device 100 shown in FIG. 1. In some examples, the LSI device 200 may also include other physiological sensors.

In the example shown in FIG. 2, the emitting device 250 includes at least one light source 260, and the sensing device 252 includes at least one detector 262 (e.g., a light sensor). In some examples, the sensing device 252 may include more than two detectors. In some examples, emitting device 250 and sensing device 252 may be separate device configured to be separately positioned, e.g., within separate nasal passages, ear canals, or along a scalp at differing positions of a patient. In other examples, emitting device 250 and sensing device 252 may be part of a single device (not shown), e.g., a device including the at least one light source 260 and the at least one detector 262.

The light source 260 may be configured to emit photonic signals having coherent light (e.g., one wavelength of light) into a subject's tissue. For example, the light source 260 may include a laser source for emitting light into the tissue of a subject to generate detectable scattering of light. The light source 260 may include any number of light sources with any suitable characteristics. In examples in which an array of sensors is used in place of the sensing device 250, each sensing device may be configured to emit the same wavelength.

The detector 262 may be chosen to be specifically sensitive to the chosen targeted energy spectrum of the light source 260. In some examples, the detector 262 may be configured to detect the intensity of light that has been scattered by tissue, as described in FIG. 1. In some examples, an array of detectors may be used and each detector in the array may be configured to detect an intensity of a single wavelength. In operation, light may enter the detector 262 after passing through the subject's tissue, including skin, bone, vasculature, and other tissue. The detector 262 may convert the intensity of the received light into an electrical signal. The light intensity may be directly related to the scattering of light from light scattering particles within the tissue that leads to the speckle contrast, as discussed with respect to FIG. 1.

After converting the received light to an electrical signal, the detector 262 may send the detection signals to the LSI device 200, which may process the detection signals and determine physiological characteristics of the patient, such as a flow value of a fluid (e.g., blood), an image of a plurality of flow values within vasculature of the tissue of the patient, the occurrence of an ischemic stroke, the occurrence of a hemorrhagic stroke, or the like. In some examples, one or more of the detection signals are preprocessed by the sensing device 252 before being transmitted to the LSI device 200.

The control circuitry 245 may be coupled to the light drive circuitry 240, the front-end processing circuitry 216, and the back-end processing circuitry 214, and may be configured to control the operation of these components. In some examples, the control circuitry 245 is configured to provide timing control signals to coordinate their operation. For example, the light drive circuitry 240 may generate one or more light drive signals, which may be used to turn on and off the light source 260, based on the timing control signals provided by the control circuitry 245. The front-end processing circuitry 216 may use the timing control signals to operate synchronously with the light drive circuitry 240. For example, the front-end processing circuitry 216 may synchronize the operation of an analog-to-digital converter and a demultiplexer with the light drive signal based on the timing control signals. In addition, the back-end processing circuitry 214 may use the timing control signals to coordinate its operation with the front-end processing circuitry 216.

The light drive circuitry 240, as discussed above, may be configured to generate a light drive signal that is provided to the light source 260 of the emitting device 250. The light drive signal may, for example, control the intensity of the light source 260 and the timing of when the light source 260 is turned on and off. In some examples, the light drive circuitry 240 provides one or more light drive signals to the light source 260. In examples in which the light source 260 is configured to emit two or more wavelengths of light, the light drive signal may be configured to control the operation of each wavelength of light. The light drive signal may comprise a single signal or may comprise multiple signals (e.g., one signal for each wavelength of light).

The front-end processing circuitry 216 may perform any suitable analog conditioning of the detector signals. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. The conditioned analog signals may be processed by an analog-to-digital converter of circuitry 216, which may convert the conditioned analog signals into digital signals. The front-end processing circuitry 216 may operate on the analog or digital form of the detector signals to separate out different components of the signals. The front-end processing circuitry 216 may also perform any suitable digital conditioning of the detector signals, such as low pass, high pass, band pass, notch, averaging, or any other suitable filtering, amplifying, performing an operation on the signal, performing any other suitable digital conditioning, or any combination thereof. The front-end processing circuitry 216 may decrease the number of samples in the digital detector signals. In some examples, the front-end processing circuitry 216 may also remove dark or ambient contributions to the received signal.

The back-end processing circuitry 214 may include the processing circuitry 210 and the memory 220. The processing circuitry 210 may include an assembly of analog or digital electronic components and may be configured to execute software, which may include an operating system and one or more applications, as part of performing the functions described herein with respect to, e.g., the processing circuitry 110 of FIG. 1. The processing circuitry 210 may receive and further process physiological signals received from the front-end processing circuitry 216. For example, the processing circuitry 210 may determine one or more physiological parameter values based on the received physiological signals. For example, the processing circuitry 210 may compute one or more of blood flow metrics and/or diagnostic metrics from the LSI signal.

The processing circuitry 210 may perform any suitable signal processing of a signal, such as any suitable band-pass filtering, adaptive filtering, closed-loop filtering, any other suitable filtering, and/or any combination thereof. The processing circuitry 210 may also receive input signals from additional sources not shown. For example, the processing circuitry 210 may receive an input signal containing information about treatments provided to the subject from the user interface 230. Additional input signals may be used by the processing circuitry 210 in any of the determinations or operations it performs in accordance with the back-end processing circuitry 214 or the LSI device 200.

The processing circuitry 210 is an example of the processing circuitry 110 and is configured to perform the techniques of this disclosure. For example, the processing circuitry 210 is configured to receive signals indicative of the speckle contrast from patient tissue and determine blood flow metrics or other values indicative of perfusion and vascular function.

The memory 220 may include any suitable computer-readable media capable of storing information that can be interpreted by the processing circuitry 210. In some examples, the memory 220 may store light source and detection functions, signal processing instructions, LSI signal processing instructions, blood flow metric calculation instructions, generated patient data, and the like. The back-end processing circuitry 214 may be communicatively coupled with the user interface 230 and the communication interface 290.

The user interface 230 may include the input device 234, the display 232, and the speaker 236 in some examples. The user interface 230 is an example of user interface 130 shown in FIG. 1, and the display 232 is an example of the display 132 shown in FIG. 1. The user interface 230 may include, for example, any suitable device such as one or more medical devices (e.g., a medical monitor that displays various physiological characteristics and/or parameters, a medical alarm, or any other suitable medical device that either displays physiological parameters or uses the output of the back-end processing 214 as an input), one or more display devices (e.g., monitor, personal digital assistant (PDA), mobile phone, tablet computer, clinician workstation, any other suitable display device, or any combination thereof), one or more audio devices, one or more memory devices, one or more printing devices, any other suitable output device, or any combination thereof.

The input device 234 may include one or more of any type of user input device such as a keyboard, a mouse, a touch screen, buttons, switches, a microphone, a joy stick, a touch pad, or any other suitable input device or combination of input devices. In other examples, the input device 234 may be a pressure-sensitive or presence-sensitive display that is included as part of the display 232. The input device 234 may also receive inputs to select a model number of the sensing device 250. In some examples, the processing circuitry 210 may determine the type of presentation for the display 232 based on user inputs received by the input device 234. For example, the processing circuitry 210 may be configured to present, via the display 232, a graphical user interface.

The communication interface 290 may enable the LSI device 200 to exchange information with other external or implanted devices. The communication interface 290 may include any suitable hardware, software, or both, which may allow the LSI device 200 to communicate with electronic circuitry, a device, a network, a server or other workstations, a display, or any combination thereof.

The components of the LSI device 200 that are shown and described as separate components are shown and described as such for illustrative purposes only. In some examples the functionality of some of the components may be combined in a single component. For example, the functionality of the front end processing circuitry 216 and the back-end processing circuitry 214 may be combined in a single processor system. Additionally, in some examples the functionality of some of the components of the LSI device 200 shown and described herein may be divided over multiple components. For example, some or all of the functionality of the control circuitry 245 may be performed in the front end processing circuitry 216, in the back-end processing circuitry 214, or both. In other examples, the functionality of one or more of the components may be performed in a different order or may not be required. In some examples, all of the components of the LSI device 200 can be realized in processor circuitry. In one example, the LSI device 200 includes the control circuitry 245 that includes all functionality described herein with respect to the front end processing circuitry 216 and the back end processing circuitry 214.

Although transmission-based laser speckle imaging is generally described herein and, in many cases, may provide appropriate physiological characteristic readings within a volume of tissue, reflectance-based laser speckle imaging (or any contribution of both) within a region can also offer valuable physiological characteristic information. Reflectance-based laser speckle imaging, which still provides physiological characteristics, may be employed by a system, such as the device 100 or LSI device 200, when the volume of tissue is too thick to adequately pass light through. In this manner, device 100, LSI device 200, or another device may obtain LSI signals from reflectance-based sensor configurations in some examples and generate physiological characteristics as described herein.

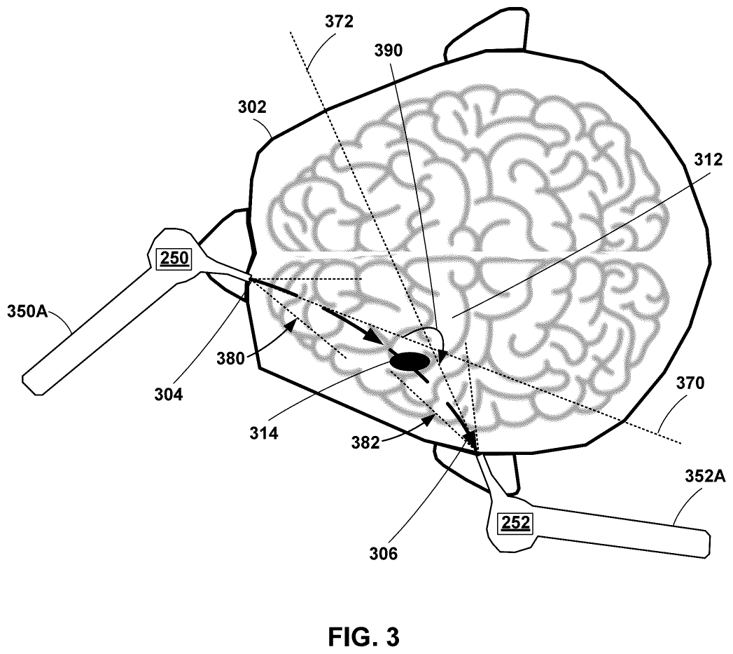

FIGS. 3-10 are a conceptual diagrams illustrating example positioning of emitting device 250 and sensing device 252 of a laser speckle imaging system, such as LSI device 200, relative to anatomy of a patient 302 and relative to a physiological characteristic of the patient 302. In the examples shown, emitting device 250 is housed in a nasoscope (or a rhinoscope, or a nasal endoscope) or an otoscope, and sensing device 252 is housed in a separate nasoscope or otoscope. In this manner, although some elements of an LSI system may be housed in physically separate devices or housings, the functionality may be similar to detection device 100 or LSI device 200.

In the example shown in FIG. 3, emitting device 250 is housed in nasoscope 350A configured to be positioned within a nasal passage of patient 302 such that light source 260 may emit coherent light 304 into tissue 312 of patient 302, e.g., into a head and/or brain of patient 302. For example, nasoscope 350A includes an insertion portion configured to be positioned at least partially within a nasal cavity and/or passage of patient 302 and that enables emitting device 250 and light source 260 to emit coherent light 304 from nasoscope 350A into tissue 312 from within the nasal passage. In some examples, the insertion portion may comprise an elongated, tubular body configured to fit within a nasal passage and may include one or more optical components, e.g., lenses, windows, filters, gratings, optical fibers, prisms, polarizers, or the like, configured to direct and/or condition coherent light 304 to be emitted into tissue 312. Nasoscope 350A may include a power source, e.g., wired power and/or a battery. Nasoscope 350A may include control circuitry, processing circuitry, and/or a wired or wireless telemetry system, e.g., to communicate with light drive circuitry 240.

Sensing device 252 is housed in otoscope 352A configured to be positioned within an ear canal of patient 302 such that detector 262 may sense and/or detect resulting light 306 from tissue of patient 302, e.g., from a head and/or brain of patient 302. For example, otoscope 352A includes an insertion portion configured to be positioned at least partially within an ear canal and/or passage of patient 302 and that enables sensing device 252 and detector 262 to detect resulting light 306 from tissue 312 from within the ear canal. In some examples, the insertion portion may comprise an elongated, tubular body configured to fit within an ear canal and may include one or more optical components, e.g., lenses, windows, filters, gratings, optical fibers, prisms, polarizers, or the like, configured to direct and/or condition resulting light 306 to be detected. Otoscope 352A may include a power source, e.g., wired power and/or a battery. Otoscope 352A may include control circuitry, processing circuitry, and/or a wired or wireless telemetry system, e.g., to communicate with front end processing circuitry 216.

In the example shown, light source 260 is configured to emit coherent light 304 from the nasal passage, and detector 262 is configured to detect resulting light 306 via the ear canal of patient 302. The resulting light 306 is generated by coherent light 304 passing through and/or interacting with at least a portion of tissue 312 vasculature and/or the brain). For example, resulting light 306 is generated by coherent light 304 being transmitted, reflected, absorbed, and/or scattered (forward and/or backward scatter) by tissue 312 as the coherent light 304 propagates from light source 260 to detector 262 (FIG. 2). Coherent light 304 may be changed and/or altered by the tissue 312, e.g., in amplitude, degree of coherence, or the like, thereby generating or forming resulting light 306. Detector 262 may be configured to detect resulting light 306 via the ear canal and generate a laser speckle image of tissue 312. The laser speckle image may be representative and/or indicative of at least a portion of tissue 312, e.g., the portion through which coherent light 304 propagates and interacts with.

In some examples, the laser speckle image generated by detector 262 based on detecting resulting light 306 may indicate, or include information regarding, a physiological characteristic of at least a portion of tissue 312. For example, processing circuitry of nasoscope 350A, otoscope 352A, and/or processing circuitry 210 may receive the laser speckle image from detector 262 and generate a speckle contrast signal based on the laser speckle image. The processing circuitry of nasoscope 350A, otoscope 352A, and/or processing circuitry 210 may determine a physiological characteristic of patient 302 and/or at least a portion of tissue 312 based on the speckle contrast signal, and output a representation of the determined physiological characteristic. In some examples, processing circuitry of nasoscope 350A, otoscope 352A, and/or processing circuitry 210 may output the physiological characteristic to user interface 230, e.g., display 232 and/or speaker 236.

In some examples, the physiological characteristic may comprise a flow value of a fluid (e.g., a blood flow value), an average fluid flow value averaged over an imaged area and/or time, an image of plurality of flow values within vasculature of tissue 312 indicative of normal, reduced, or no fluid flow, an occurrence of an ischemic stroke, and occurrence of a hemorrhagic stroke, or the like. For example, tissue 312 may include stroke location 314. Stroke location 314 may be a portion of tissue 312 affected by an ischemic stroke, e.g., a location in which brain cells are subjected to reduced oxygen delivery or in which there is reduced or no blood flow through vasculature, or a portion of tissue 312 affected by a hemorrhagic stroke, e.g., a location in which there is low or no blood flow through a blood vessel due to a rupture of the blood vessel, or an excess of blood that has bled out of the vessel and is no longer moving, or the like. In some examples, the physiological characteristic comprises stroke location 314, which may include one or both of a location of a stroke within patient 302 and its severity.

In the example shown, LSI device 200 may improve the signal and/or signal to noise ratio of resulting light 306 from which the laser speckle contrast image is generated and from which the physiological characteristic is determined by positioning emitting device 250 within nasoscope 350A such that coherent light is emitted closer to portions of tissue 312 of interest (e.g., portions in which strokes may occur such as stroke location 314) with reduced or no relatively dense tissue (e.g., bone) in the path, e.g., thereby increasing the amount of coherent light 304 interacting with tissue 312 of interest. Also, LSI device 200 may improve the signal and/or signal to noise ratio of resulting light 306 by positioning sensing device 252 within otoscope 352A such that detector 262 receives resulting light 306 closer to portions of tissue 312 of interest (e.g., portions in which strokes may occur such as stroke location 314) with reduced or no relatively dense tissue (e.g., bone) in the path, e.g., thereby increasing the amount of resulting light 306 detected.

In the example shown, light source 260 of emitting device 250 defines a source optical axis 370 defining an emission direction of light source 250 and coherent light 304, and detector 262 of sensing device 252 defines a detector optical axis 372 defining a viewing direction of detector 262. Light source 260 also defines emission angles 380 comprising a solid angle into which light source 260 emits light about source optical axis 370, and detector 262 also defines detector angles 382 comprising a solid angle from which detector 26 detects light about detector optical axis 372. In some examples, light source 260 and detector 262 are positioned such that an angle 390 between the detector optical axis 372 and the source optical axis 70 is greater than 45 degrees and less than 270 degrees. For example, detector 262 is positioned to detect resulting light 306 that “transmits” or forward scatters through tissue 312, e.g., where angle 390 being zero degrees corresponds to light source 260 and detector 262 facing the same direction and angle 390 being 180 degrees corresponds to light source 260 and detector 262 facing each other, e.g., opposite directions. In the example shown, light source 260 and detector 262 are positioned via nasoscope 350A and otoscope 352A such that angle 390 is greater than 45 degrees and less than 270 degrees, e.g., about 225 degrees as shown. In other examples, light source 260 and detector 262 are positioned such that an angle 390 between the detector optical axis 372 and the source optical axis 370 is less than or equal to 90 degrees or greater than or equal to 270 degrees (not shown). For example, detector 262 may be positioned to detect resulting light 306 that is “reflects” or back scatters through tissue 312.

In some examples, nasoscope 350A and otoscope 352A are configured to be separated from each other by a threshold distance. For example, separating nasoscope 350A and otoscope 352A separates light source 260 and detector 262 by the threshold distance such that detector 262 detects resulting light 306 coming from coherent light 304 that has penetrated through a greater depth within tissue 312, e.g., as opposed to most of the LSI signal being backscatter from a relatively shallow portion of tissue 312 if nasoscope 350A and otoscope 352A were right next to each other. In some examples, the threshold distance corresponds to any or all of a depth within tissue 312 and/or an amount or volume of tissue 312 that coherent light 304 may interact with. For example, light source 260 may be configured to emit the coherent light 304 within a range of emission angles 380, e.g., emission angle 380 may subtend a solid angle with a beam divergence of at least 15 degrees along one axis (e.g., having a solid angle apex angle of at least 7.5 degrees, or of at least 15 degrees) with nasoscope 350A positioning light source 260 within a nasal passage of patient 302. Otoscope 352A is positioned within an ear canal of patient 302 and separated by a threshold distance from nasoscope 350A such that coherent light 304 must propagate through a threshold depth distance through tissue 312 thereby generating resulting light 306 to be detected by detector 262.

In some examples, the nasoscope 350A may be positioned in the other nasal passage of patient 302, and/or otoscope 352A may be positioned in the other ear canal of patient 302.

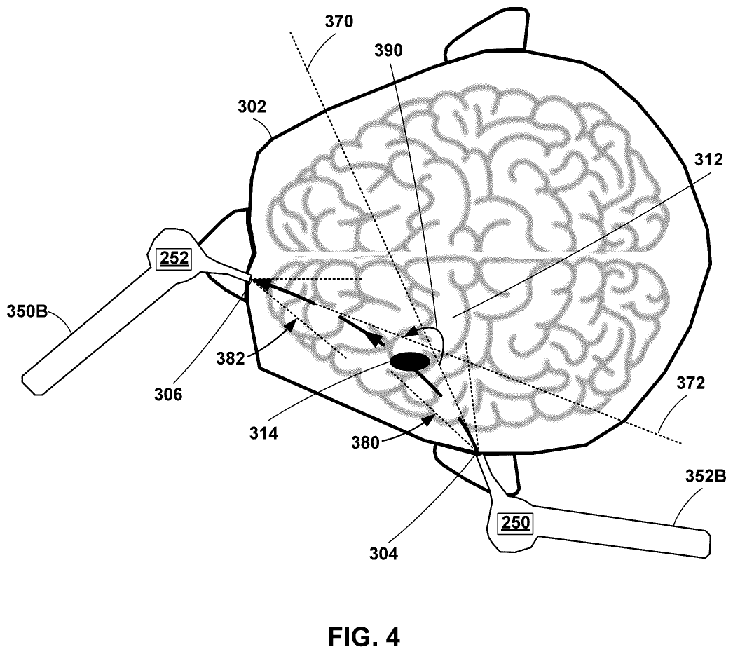

In some examples, emitting device 250 and sensing device 252 may be switched between the nasoscope and the otoscope. For example, as shown in FIG. 4, emitting device 250 may be housed in otoscope 352B configured to be positioned within an ear canal of patient 302 such that light source 260 may emit light 304 into tissue of patient 302 via the ear canal. Sensing device 252 may be housed in nasoscope 350B configured to be positioned within a nasal passage of patient 302 such that detector 262 may sense and/or detect light 304 from tissue of patient 302 via the nasal passage, and light 304 may traverse from the ear canal to the nasal passage. In some examples, the nasoscope 350B may be positioned in the other nasal passage of patient 302, and/or otoscope 352B may be positioned in the other ear canal of patient 302. Nasoscope 350B may be substantially similar to nasoscope 350A except that the nasoscope 350B includes sensing device 252 rather than emitting device 250, and otoscope 352B may be substantially similar to otoscope 352A except that the otoscope 352B includes emitting device 250 rather than sensing device 252. For example, otoscope 352B may include an insertion portion configured to be positioned at least partially within an ear canal of patient 302 and that enables emitting device 250 and light source 260 to emit coherent light 304 from otoscope 350B into tissue 312 from within the nasal passage. In some examples, the insertion portion may comprise an elongated, tubular body configured to fit within the ear canal and may include one or more optical components, e.g., lenses, windows, filters, gratings, optical fibers, prisms, polarizers, or the like, configured to direct and/or condition coherent light 304 to be emitted into tissue 312. Otoscope 352B may include control circuitry, processing circuitry, and/or a wired or wireless telemetry system, e.g., to communicate with light drive circuitry 240. Nasoscope 352B may include an insertion portion configured to be positioned at least partially within a nasal cavity and/or passage of patient 302 and that enables sensing device 252 and detector 262 to detect resulting light 306 from tissue 312 from within the nasal passage. In some examples, the insertion portion may comprise an elongated, tubular body configured to fit within the nasal passage and may include one or more optical components, e.g., lenses, windows, filters, gratings, optical fibers, prisms, polarizers, or the like, configured to direct and/or condition resulting light 306 to be detected. Nasoscope 352B may include control circuitry, processing circuitry, and/or a wired or wireless telemetry system, e.g., to communicate with front end processing circuitry 216.

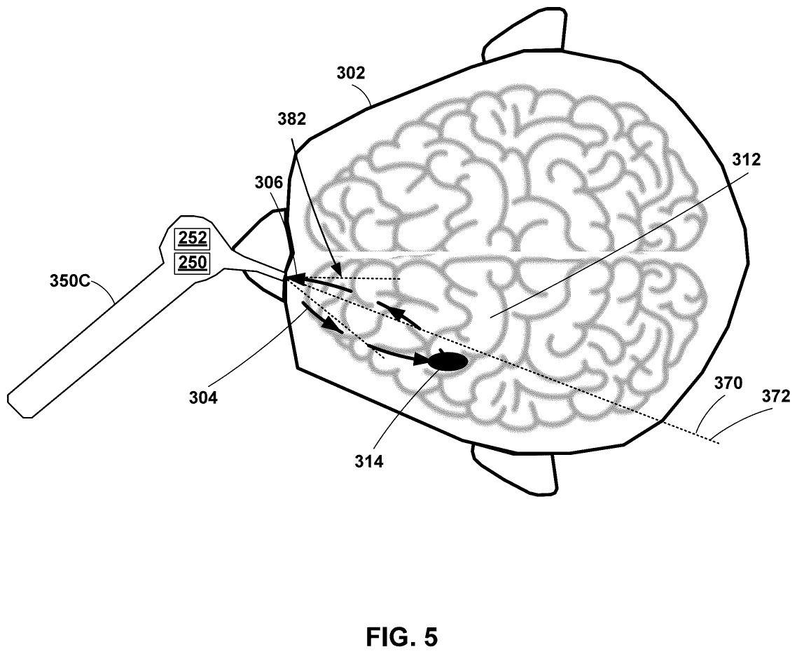

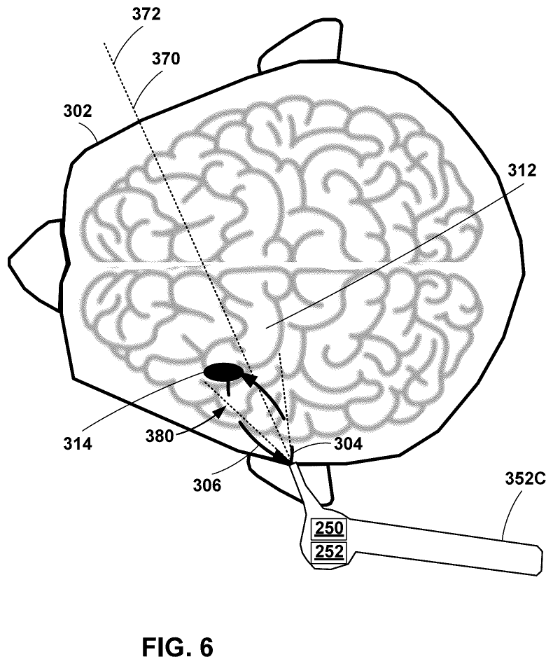

In some examples, both emitting device 250 and sensing device 252 may be housed in nasoscope 350C configured to be positioned within a nasal passage of patient 302 such that light source 260 may emit light 304 into tissue of patient 302 via the nasal passage and detector 262 may sense and/or detect resulting light 306 from tissue of patient 302 via the same nasal passage, e.g., as shown in FIG. 5. For example, coherent light 304 may propagate from the nasal passage to the tissue 312 and back to the nasal passage (e.g., backscatter) as resulting light 306. In some examples, both emitting device 250 and sensing device 252 may be housed in otoscope 352C configured to be positioned within an ear canal of patient 302 such that light source 260 may emit coherent light 304 into tissue of patient 302 via the ear canal and detector 262 may sense and/or detect resulting light 306 from tissue of patient 302 via the same ear canal, e.g., as shown in FIG. 6. For example, coherent light 304 may traverse from the ear canal to the tissue and back to the ear canal (e.g., backscatter) as resulting light 306. Nasoscope 350C may be substantially similar to nasoscope 350A except that the nasoscope 350C includes both emitting device 250 and sensing device 252, and otoscope 352C may be substantially similar to otoscope 352A except that the otoscope 352C includes both emitting device 250 and sensing device 252. For example, nasoscope 350C and otoscope 352C may include an insertion portion configured to enable light source 260 to emit coherent light 304 into tissue 312 and to enable detector 262 to detect resulting light 306 from tissue 312, as described above with nasoscope 350A and 350B and otoscope 352A and 352C, and nasoscope 350C and otoscope 352C may include control circuitry, processing circuitry, and/or a wired or wireless telemetry system, e.g., to communicate with light drive circuitry 240 and front end processing circuitry 216.

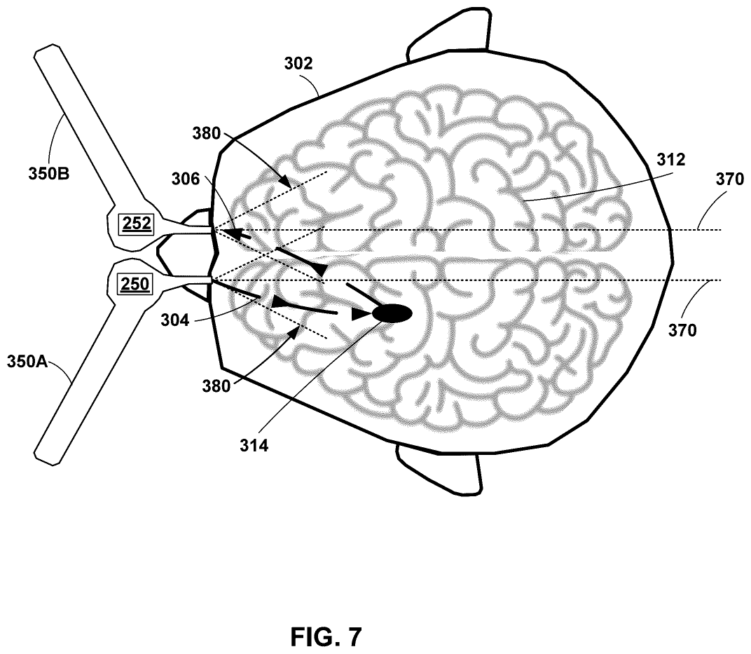

In some examples, emitting device 250 and sensing device 252 may be housed in separate, respective nasoscopes 350A, 35B each configured to be positioned within a different nasal passage of patient 302 such that light source 260 may emit light 304 into tissue of patient 302 via one nasal passage and detector 262 may sense and/or detect resulting light 306 from tissue of patient 302 via the other nasal passage, e.g., as shown in FIG. 7. For example, coherent light 304 may propagate from the nasal passage to the tissue 312 and back to the other nasal passage (e.g., backscatter) as resulting light 306.

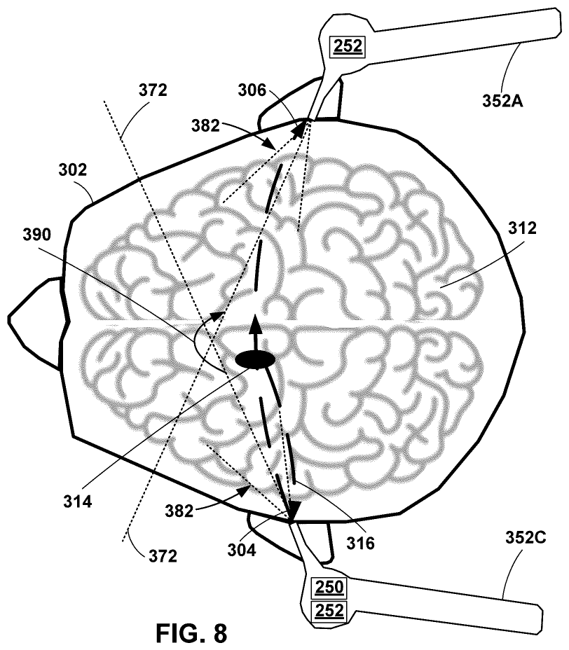

In some examples, emitting device 250 and sensing device 252 may be housed in separate, respective otoscopes 352A, 352C each configured to be positioned within a different ear canal of patient 302 such that light source 260 may emit light 304 into tissue of patient 302 via one ear canal and detector 262 may sense and/or detect resulting light 306 from tissue of patient 302 via the other ear canal, e.g., as shown in FIG. 8. For example, coherent light 304 may propagate from the ear canal to the tissue 312 and to the other ear canal (e.g., transmission and/or forward scatter) as resulting light 306. Alternatively or in addition, coherent light 304 may propagate from the ear canal to the tissue 312 and back to the same ear canal (e.g., backscatter) as resulting light 316 (which may be the same or different from resulting light 306, e.g., having propagated along a different optical path to reach a detector 262). In the example shown, one otoscope 352C may include both emitting device 250 and sensing device 252, and the other otoscope 352A may include sensing device 252.

In some examples, LSI device 200 may include one or two nasoscopes 350A, 350B, or 350C, which may include one or both of emitting device 250 and sensing device 252, and LSI device 200 may additionally or alternatively include one or two of otoscopes 352A, 352B, or 352C, which may include one or both of emitting device 250 and sensing device 252.

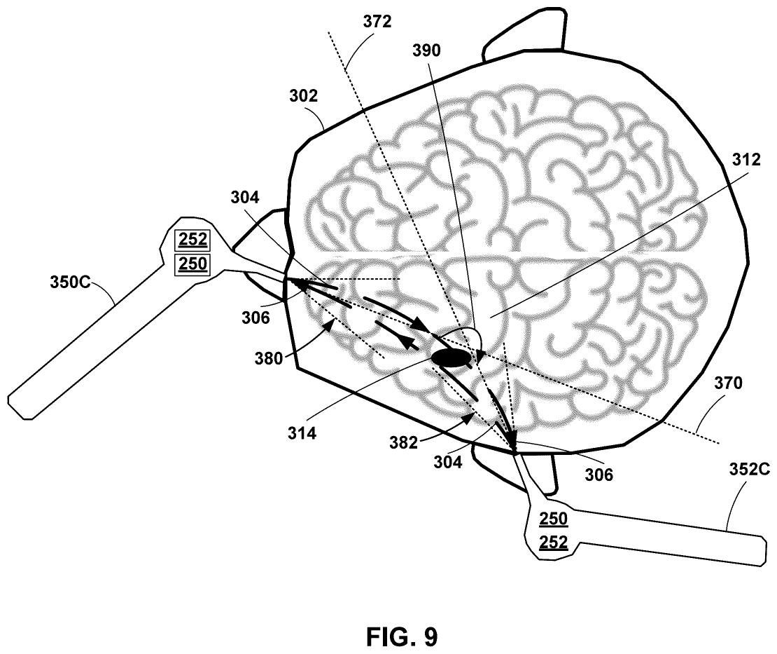

In some examples, LSI device 200 may include a plurality of emitting devices 250, light sources 260, sensing devices 252, and detectors 262. In the example shown in FIG. 9, both an emitting device 250 and a sensing device 252 may be housed in nasoscope 350C and another emitting device 250 and another sensing device 252 may be housed in otoscope 352C. A detector 262 within nasoscope 350C may then detect light 304 propagating from light source 260 from nasoscope 350C (e.g., via backscatter from tissue) and/or light 304 propagating from light source 260 within otoscope 352C (e.g., via transmission and/or forward scattering from tissue). Similarly, in sequence or concurrently, a detector 262 within otoscope 352C may detect light 304 propagating from light source 260 from otoscope 352C (e.g., via backscatter from tissue) and/or light 304 propagating from light source 260 within nasoscope 350C (e.g., via transmission and/or forward scattering from tissue).

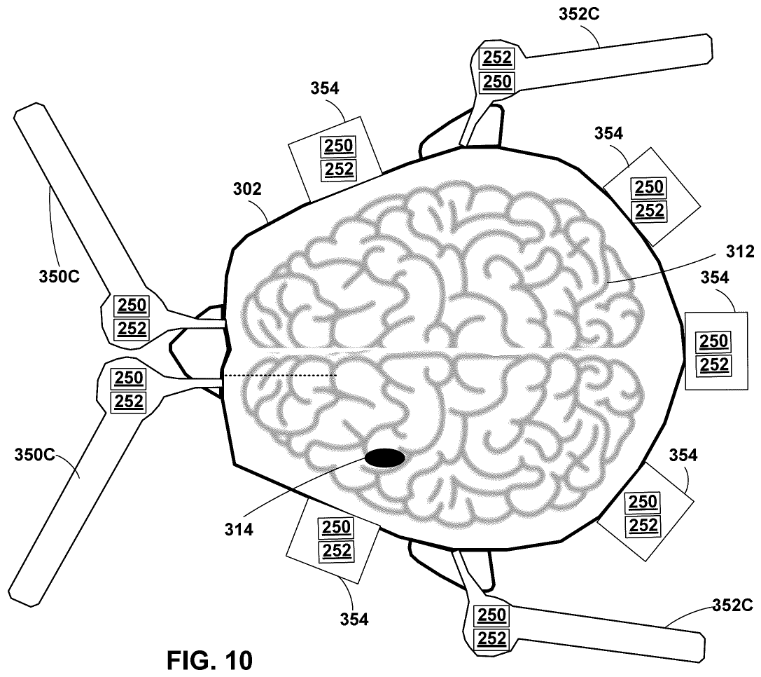

Additionally or alternatively, one or more emitting devices 250 and/or sensing devices 252 may be housed in one or more scalp housings 354 configured to be positions at a scalp of patient 302 such that light source 260 may emit coherent light 304 into tissue 312 of patient 302 and/or such that detector 262 may sense and/or detect resulting light 306 from tissue of patient 302, as shown in FIG. 10. For example, scalp housings 354 may be configured to position light source 260 to emit coherent light 304 through the skull of patient 302 into tissue 312. Scalp housings 354 may be configured to position detector 262 to detect resulting light 306 through the skull of patient 302 from tissue 312. In the example shown, one or more of any of nasoscopes 350, otoscopes 352, and scalp housings 354 may be configured to position one or more of emitting device 250 and one or more sensing device 252 in one or more nasal passage, ear canal, and/or one or more positions about the scalp of patient 302 in any combination, the light sources 260 of which may emit light 304 into tissue 312 and the detectors 262 of which may detect resulting light 306 from tissue 312 (e.g., originating from any of the one or more light sources 260), concurrently and/or in any sequence.

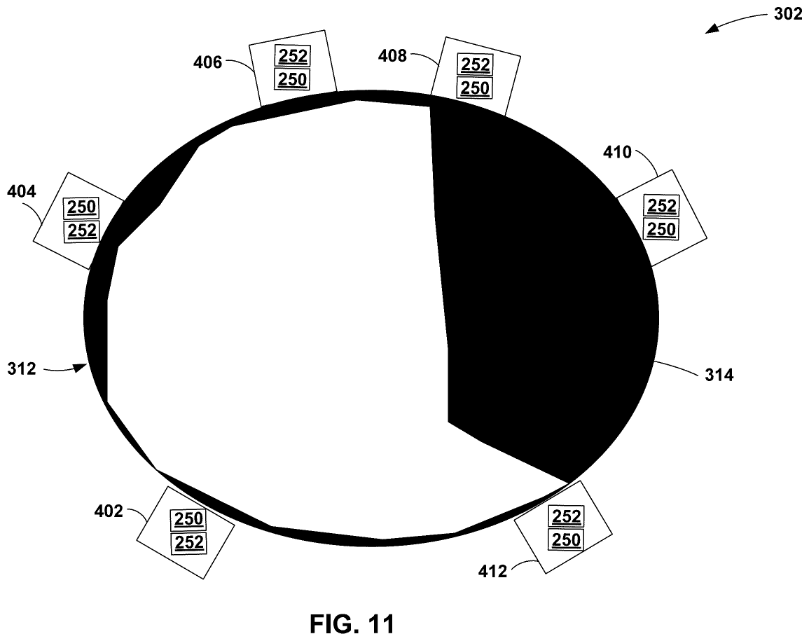

In some examples, LSI device 200 may include a plurality of emitting devices 250 and sensing devices 252 housed within one or more nasoscopes, otoscopes, and/or scalp housings and configured to map one or more physiological characteristics of an organ, such as a brain, of patient 302. FIG. 11 is a conceptual diagram illustrating another example positioning emitting devices 250 and sensing devices 252 relative to anatomy of patient 302 to map a portion, or the whole, of an organ of patient 302, e.g., a brain of patient 302. In the example shown, LSI device 200 may include a plurality of scalp housings 402-412, each of which is configured to position an emitting device 250 and a sensing device 252 configured to emit coherent light 304 into a portion of the brain and to detect resulting light 306 from a portion of the brain, e.g., to measure one or more physiological characteristics of a plurality of portions of the brain via LSI.

In the example shown, scalp housing 402 is configured to position emitting device 250 and sensing device 252 to measure posterior cerebral arteries of the left hemisphere of the brain, scalp housing 404 is configured to position emitting device 250 and sensing device 252 to measure middle cerebral arteries of the left hemisphere of the brain, and scalp housing 406 is configured to position emitting device 250 and sensing device 252 to measure anterior cerebral arteries of the left hemisphere of the brain. Similarly, scalp housing 412 is configured to position emitting device 250 and sensing device 252 to measure posterior cerebral arteries of the right hemisphere of the brain, scalp housing 410 is configured to position emitting device 250 and sensing device 252 to measure middle cerebral arteries of the right hemisphere of the brain, and scalp housing 408 is configured to position emitting device 250 and sensing device 252 to measure anterior cerebral arteries of the right hemisphere of the brain. In the example shown, tissue 312 includes location 314 which includes a physiological characteristic and/or condition of patient 302, e.g., stroke location 314. LSI device 200 may determine that emitting devices 250 and sensing devices 252 of scalp housings 402-408 and 412 measure LSI indicating normal physiological characteristics (e.g., blood flow) of their respective portions of tissue 312 (the brain of patient 302), and that emitting device 250 and sensing device 252 of scalp housing 410 measures an LSI indicating an abnormal physiological characteristic (e.g., low or no blood flow) of a right middle cerebral artery. From the measurements, LSI device 200 may determine the location and/or severity of a condition, e.g., ischemic stroke, hemorrhagic stroke, damaged arteries and/or veins, damaged tissue, a tumor location and/or size, or the like, based on the LSI measurements and/or asymmetry of physiological characteristics (e.g., blood flow) determined via LSI measurements.

In some examples, one or more emitting devices 250 and sensing devices 252 may be positioned to measure specific portions of anatomy of patient 302. For example, one or more light sources 260 may be configured to be positioned to emit coherent light 304 to a Circle of Willis, a proximal vessel, a carotid artery, or any suitable vasculature of the head and/or neck of patient 302, e.g., via positioning of one or more of any of nasoscope 350, otoscope 352, and/or scalp housings 354. Similarly, one or more detectors 262 may be configured to be positioned to detect resulting light 306 from a Circle of Willis, a proximal vessel, a carotid artery, or any suitable vasculature of the head and/or neck of patient 302, e.g., via positioning of one or more of any of nasoscope 350, otoscope 352, and/or scalp housings 354.

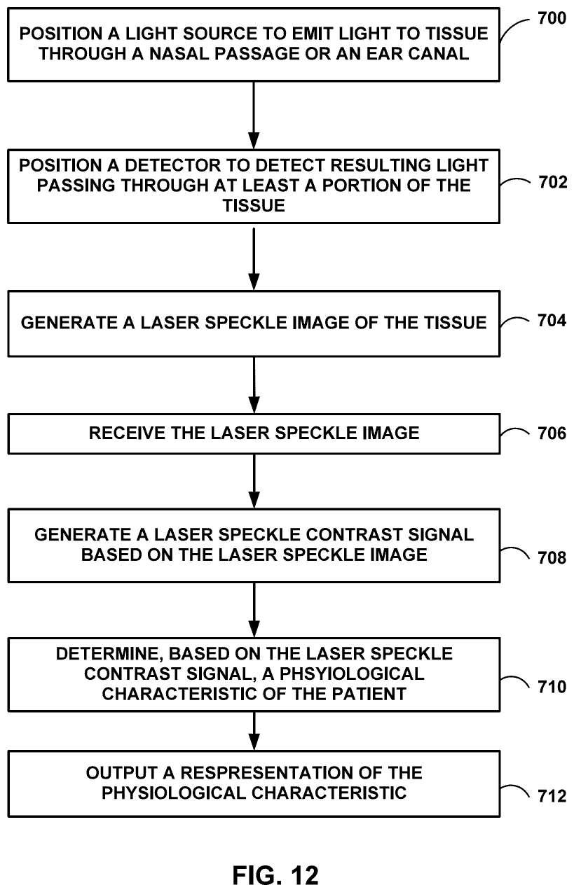

FIG. 12 is a flow diagram illustrating example techniques for determining a physiological characteristic of a patient 302 using a laser speckle imaging system. FIG. 12 is described with respect to processing circuitry 110 of the blood flow detection device 100 (FIG. 1), processing circuitry 210 of LSI device 200 (FIG. 2), nasoscope 350, otoscope 352, scalp housings 354, emitting device 250 and light source 260, and sensing device 252 and detector 262 (FIGS. 3-11). In other examples, other processing circuitry, alone or in combination with the processing circuitry 110 or 210, may perform any part of the techniques of FIG. 12. The technique of FIG. 12 may be automatically performed by a system or have one or more elements performed by a human user, such as a clinician. In this manner, the technique may be described in specific portions with reference to a specific user or device configured to perform those portions of the technique.

In the example of FIG. 12, a user may position light source 260 to emit coherent light 304 to tissue 312 of a patient 302 through at least one of a nasal passage or an ear canal of patient 302 (700). For example, a user may position nasoscope 350 within a first nasal passage of patient 302 (as shown in FIG. 3). The user may position detector 262 to detect resulting light 306 passing through at least a portion of the tissue 312 (702). For example, the user may position otoscope 352 within a first ear canal of patient 302 as shown in (FIG. 3).

The detector 262 may generate, based on resulting light 306, a laser speckle image of tissue 312 (704). In some examples, the laser speckle image is representative of the portion of the tissue 312 interacting with the coherent light traveling through at least the nasal passage or the ear canal of the patient 302.

Processing circuitry 110 and/or 210 may receive the laser speckle image from detector 262 (706), and processing circuitry 110 and/or 210 may generate a speckle contrast signal based on the laser speckle image (708).

Processing circuitry 110 and/or 210 may determine, based on the speckle contrast signal, a physiological characteristic of patient 302 (710), and output a representation of the physiological characteristic of patient 302 (712). For example, processing circuitry 110 and/or 210 may determine at least one of a flow value of a fluid (e.g., blood), an image of a plurality of flow values within vasculature of the tissue of the patient 302, an ischemic stroke, or a hemorrhagic stroke, based on the speckle contrast signal. In some examples, positioning the light source 260 and detector 262 within a nasal passage and/or ear canal of the patient 302, separated by a threshold distance, enables LSI device 200 to emit coherent light 304 and detect resulting light 306 having propagated through a depth of tissue 312 with in improved signal and/or signal to noise ratio from which to determine a physiological characteristic of patient 302 occurring and/or localized at a relatively deeper position within tissue 312, e.g., without introducing light source 260 or detector 262 into tissue 312. Processing circuitry 110 and/or 210 may output a representation of the determined physiological characteristic to user interface 230, e.g., an image, character, number, or visual indicator via display 232, or an audible indicator and/or warning via speaker 236. In some examples, processing circuitry 110 and/or 210 may output a representation of the determined physiological characteristic as a color map indicating blood vessels and flow velocities within those vessels, or a representative value such as the average blood flow rate in the interrogated tissue, or a representative waveform showing the blood flow over time in the interrogated tissue, or a diagnosis of whether the patient is having an ischemic stroke based on the LSI data. In some examples, processing circuitry 110 and/or 210 may output, to user interface 230, a recommendation based on the determined characteristic.

In some examples, a user (e.g., a clinician) may diagnose an ischemic stroke using LSI device 200 and/or the techniques above. For example, LSI device 200 may be used to generate a representation of the determined physiological characteristic comprising a detailed image of neurovasculature of patient 302, e.g., comparable to a CT image or scan, and provide blood flow measurements within interrogated areas of the neurovasculature. This information (e.g., image) may be used to accurately diagnose an ischemic stroke and identify a location of an occlusion.

In some examples, a user or clinician may accurately diagnose a stroke using less data than needed to form a full detailed image, and meaningful information about the stroke location may be determined using less data than a full detailed image. For example, by applying LSI at any region of the brain, it is possible to determine the average blood flow in the measured volume. If the average blood flow value is outside of the healthy range, LSI device 200 may determine an occurrence of a stroke, or LSI device 200 may output a representation indicative of the average blood flow value being outside of the healthy range from which a user and/or clinician may determine an occurrence of a stroke. In some examples, LSI device 200 may be used to determine the average blood flow from the brain's left hemisphere and right hemisphere, and the average blood flow from the left and right hemispheres may be compared against each other, e.g., by LSI device 200 and/or a user/clinician using LSI device 200. If the difference between the hemispheres exceeds a certain value, the imbalance maybe indicative of the occurrence of a stroke, and LSI device 200 may automatically determine the occurrence of the stroke based on the difference or a metric determined from the difference. In some examples, LSI device 200 may measure and/or determine flow dynamics within tissue of patient 302 (e.g., how the flow changes as a function of time) and may determine and/or detect an ischemic stroke based on flow dynamics and/or a metric derived from the flow dynamics.

In some examples, the user may determine a physiological characteristic of a patient 302 using a LSI device 200 at multiple regions of the brain of patient 302. LSI device 200, and/or the user, may then identify regions with less blood flow than other regions. Identifying a local region with less blood flow may improve and/or enable the user/clinician to determine a location of an occlusion, e.g., before performing any CT, MRI, or angiogram. For example, if low blood flow is detected by LSI device 200 in only the left anterior region, LSI device 200 and/or the user/clinician may determine that patient 302 has an occlusion in the anterior cerebral artery (ACA). However, if low blood flow is detected by LSI device 200 throughout the left hemisphere, LSI device 200 and/or the user/clinician may determine that an occlusion is in a proximal region such as the left internal carotid artery (ICA) and patient 302 may not have a complete Circle of Willis because blood flow from the right hemisphere does not adequately compensate for the occluded left ICA. LSI device 200 may provide information, e.g., about occlusion location and anatomy, which a user/clinician may use to plan an approach to treating ischemic stroke and may improve the timeliness and effectiveness of patient treatment.

In some examples, a user (e.g., a clinician) may diagnose other medical conditions associated with stroke using LSI device 200 and/or the techniques above. For example, alternatively or in addition to performing LSI to measure blood flow, LSI device 200 may be configured to perform other measurements using light to collect data relevant to stroke treatment. For example, LSI device 200 may be configured to perform bulk tissue or pulse oximetry to measure the oxygenation of blood within interrogated tissue or arteries, respectively. LSI device 200 may be configured to use spectroscopic techniques, e.g., detect a spectra of a plurality of wavelengths of light interrogating tissue of patient 302, e.g., via multiple light emitters configured to emit specific light frequencies and/or multiple detectors configured to detect specific light frequencies. In some examples, LSI device 200 may detect, during an ischemic stroke, oxygenated blood that does not reach a certain region of the brain, and LSI device 200 configured to determine tissue and/or pulse oximetry may improve the accuracy of the stroke diagnosis and provide information on the amount of salvageable tissue, if blood flow is restored.

In some examples, LSI device 200 may be configured to perform spectroscopic techniques to assess other pertinent tissue chromophores, such as water. For example, LSI device 200 may include light sources with emission bands at or near the optical absorption spectrum of water and may be configured to determine a degree of light absorption at specific frequencies (e.g., in isolation, or relative to other measurements). LSI device 200 may comprise detectors which are fabricated, tuned, or filtered to be sensitive to specific portions of the optical absorption spectrum of water and may be configured to determine cerebral edema. For example, during an ischemic stroke, the ion concentration and osmotic pressure in the affected tissue may change, water may enter the cells, and cell swelling may occur. LSI device 200 may improve the accuracy of a stroke diagnosis by detecting cerebral edema. LSI device 200 may be configured to measure a volume of tissue affected by edema and may provide information on the amount of salvageable tissue, if blood flow is restored.

In some examples, a user (e.g., a clinician) may diagnose a hemorrhagic stroke using LSI device 200 and/or the techniques above. For example, a hemorrhagic stroke may be associated with, or indicative of, a ruptured aneurysm. Unlike an ischemic stroke, which may be associated with, or indicative of, reduced blood flow to a region, a hemorrhagic stroke may be characterized by hyperperfusion or differing flow dynamics. LSI device 200 may be configured to measure blood flow that exceeds the healthy range, and LSI device 200 and/or a user/clinician may diagnose a hemorrhagic stroke based on the measured blood flow that exceeds the healthy range.

In some examples, LSI device 200 may be configured to detect hemorrhaged blood and/or an increase in a local fluid concentration indicative of hemorrhaged blood. In some examples, LSI device 200 may measure and/or determine flow dynamics indicative of a hemorrhagic stroke, which may improve the accuracy of the hemorrhagic stroke diagnosis.

In some examples, a user (e.g., a clinician) may diagnose a stroke before patient 302 is in a hospital using LSI device 200 and/or the techniques above. For example, an EMT may use LSI device 200 to diagnose a stroke. In some examples, an EMT may use any of nasoscope 350, otoscope 352, and/or scalp housings 354 and LSI device 200 to diagnose a stroke prior to placing the patient in an ambulance. The diagnosis may be communicated to a dispatcher, who may direct the EMT to transport patient 302 the nearest hospital with a stroke center and the stroke center may prepare the clinician and catheter lab for an incoming stroke patient.

In some examples, LSI device 200, used to provide a pre-hospital stroke diagnosis and/or determination, may reduce a time to treatment and improve treatment outcomes, improve quality of life for the patient 302, and reduce cost to the patient 302 and/or an integrated health system, e.g., reducing a hospital stay and/or post-treatment care. In some examples, LSI device 200, used to provide a pre-hospital stroke diagnosis and/or determination, may avoid or prevent a patient from being simply transported to the nearest emergency room (ER) at a hospital without a stroke center, and avoid and/or prevent the associated delay in treatment and increased cost to the patient and insurer.

In some examples, LSI device 200, used to provide a pre-hospital stroke diagnosis and/or determination, may reduce a stroke diagnosis time, which may provide identification of more stroke patients who are still within a time-window for treatment with intravenous (IV) thrombolytics or mechanical thrombectomy.

In some examples, a user (e.g., a clinician) may provide in-hospital monitoring using LSI device 200 and/or the techniques above. For example, after stroke treatment, the patient 302 may require monitoring to verify that the treatment was effective. LSI device 200 may supplement and/or replace follow-up CT, MRI, or angiogram, e.g., by measuring and/or determining if blood flow has been restored to the affected region or that the average blood flow is within normal ranges. LSI device 200 may be configured to perform a single inspection, or it may be configured to perform a continuous monitoring, e.g., via a wearable helmet (scalp housings 354) to track the patient's recovery.

LSI device 200 may improve treatment outcomes by identifying stroke recurrence quickly (such as with continuous monitoring) and may provide notification to the clinician that additional intervention may be required, LSI device 200 may reduce cost to the patient and insurer by not requiring a follow-up imaging with CT, MRI, or angiogram, and may reduce cost by identifying when the patient has adequately recovered to shorten a hospital stay.

In some examples, a user (e.g., a clinician) may provide at-home monitoring using LSI device 200 and/or the techniques above. For example, a person who suffers a stroke may be more likely to suffer another stroke after treatment. A wearable helmet (e.g., scalp housings 354) and LSI device 200 may be used to provide continuous monitoring at home after discharge. LSI device 200 may be configured to notify the patient to contact emergency responders upon detection and/or determination of a stroke by LSI device 200. A wearable helmet (e.g., scalp housings 354) and LSI device 200 may automatically contact emergency responders on behalf of the patient.

In some examples, LSI device 200 may provide data collection and analysis. For example, the measurements performed with each use of LSI device 200 may be collected by a manufacturer or a user (e.g., a clinician and/or a hospital) for use other than stroke diagnosis. The data may be used for research and development of stroke diagnostic or treatment techniques or products. The data may also be shared (e.g., with researchers, universities, or companies) to develop products and/or therapies not related to stroke.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to processing circuitry 110 and/or 210 and any other processing circuitry or electrical circuitry, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

As used herein, the term “circuitry” refers to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. For example, any of the techniques or processes described herein may be performed within one device or at least partially distributed amongst two or more devices. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache). Elements of devices and circuitry described herein, including, but not limited to, devices 100 and 200 may be programmed with various forms of software. The one or more processors may be implemented at least in part as, or include, one or more executable applications, application modules, libraries, classes, methods, objects, routines, subroutines, firmware, and/or embedded code, for example.

Select examples of the present disclosure include, but are not limited to, the following examples.

Example 1: A system including: a light source configured to emit at least quasi-coherent light to tissue of a patient, the light source configured to emit the at least quasi-coherent light through at least one of a nasal passage or an ear canal of the patient; a detector configured to detect resulting light passing through at least a portion of the tissue and generate an interference pattern image, wherein the interference pattern image is representative of the portion of the tissue interacting with the quasi-coherent light traveling through at least the nasal passage or the ear canal of the patient, and processing circuitry configured to: receive the interference pattern image from the detector; generate a speckle contrast signal based on the interference pattern image; determine, based on the speckle contrast signal or a metric derived from the speckle contrast signal, a physiological characteristic of the patient; and output a representation of the physiological characteristic of the patient.

Example 2: The system of example 1, wherein the physiological characteristic of the patient comprises at least one of a flow value of a fluid, an image of a plurality of flow values within vasculature of the tissue of the patient, an occurrence of an ischemic stroke, or an occurrence of a hemorrhagic stroke.

Example 3: The system of example 1 or example 2, wherein the light source is housed in one of a nasoscope or an otoscope.

Example 4: The system of any one of examples 1 through 3, wherein the detector comprises a detector optical axis defining a viewing direction of the detector, wherein the light source comprises a source optical axis defining an at least quasi-coherent light emission direction, wherein the detector is positioned such that an angle between the detector optical axis and the source optical axis is greater than 90 degrees and less than 270 degrees.

Example 5: The system of any one of examples 1 through 4, further comprising a first housing and a second housing, wherein the light source is disposed within the first housing, wherein the detector is disposed within the second housing different from the first housing, and wherein the first housing and the second housing are configured to be separated from each other by a threshold distance.

Example 6: The system of example 5, wherein the nasal passage is a first nasal passage, wherein the first housing is a nasoscope configured to position the light source in the first nasal passage of the patient, and wherein the second housing is configured to position the detector in at least one of a second nasal passage of the patient, the ear canal of the patient, or at a scalp of the patient.

Example 7: The system of example 5, wherein the ear canal is a first ear canal, wherein the first housing is an otoscope configured to position the light source in the first ear canal of the patient, and wherein the second housing is configured to position the detector in at least one of the nasal passage of the patient, a second ear canal of the patient, or at a scalp of the patient.

Example 8: The system of example 5, wherein the first housing is a head mounted device configured to be position the light source at a first position at a scalp of the patient, wherein the second housing is configured to position the detector in at least one of the nasal passage of the patient, the ear canal of the patient, or at a second position along the scalp of the patient, wherein the interference pattern image is representative of the portion of the tissue interacting with the quasi-coherent light traveling through the scalp of the patient.

Example 9: The system of any one of examples 1 through 8, wherein the light source is configured to emit the at least quasi-coherent light with a beam divergence of at least 15 degrees along one axis.

Example 10: The system of example 9, wherein the detector comprises a plurality of detectors positioned in at least one of the nasal passage of the patient, the ear canal of the patient, or at a scalp of the patient, wherein each detector of the plurality of detectors are separated from each other by a threshold distance.

Example 11: The system of any one of examples 1 through 10, wherein light source is configured to be positioned to emit the quasi-coherent light to a Circle of Willis or a proximal vessel within a head or neck of the patient.

Example 12: A method including: positioning a light source to emit at least quasi-coherent light to tissue of a patient through at least one of a nasal passage or an ear canal of the patient; positioning a detector to detect resulting light passing through at least a portion of the tissue; generating, by the detector and based on the resulting light, an interference pattern image, wherein the interference pattern image is representative of the portion of the tissue interacting with the quasi-coherent light traveling through at least the nasal passage or the ear canal of the patient; receiving, by processing circuitry, the interference pattern image from the detector; generating, by the processing circuitry, a speckle contrast signal based on the interference pattern image; determining, by the processing circuitry and based on the speckle contrast signal or a metric derived from the speckle contrast signal, a physiological characteristic of the patient; and outputting a representation of the physiological characteristic of the patient.

Example 13: The method of example 12, wherein the physiological characteristic of the patient comprises at least one of a flow value of a fluid, an image of a plurality of flow values within vasculature of the tissue of the patient, an ischemic stroke, or a hemorrhagic stroke.

Example 14: The method example 12 or example 13, wherein the light source is housed in at least one of a nasoscope or an otoscope.

Example 15: The method of any one of examples 12 through 14, wherein the detector comprises a detector optical axis defining a viewing direction of the detector, wherein the light source comprises a source optical axis defining a quasi-coherent light emission direction, wherein positioning the light source and positioning the detector comprises positioning the light source and positioning the detector such that an angle between detector optical axis and the source optical axis is greater than 90 degrees and less than 270 degrees.

Example 16: The method of any one of examples 12 through 15, wherein the light source is disposed within a first housing, wherein the detector is disposed within a second housing different from the first housing, wherein positioning the light source and positioning the detector comprises positioning the first housing separated from the second housing by a threshold distance.

Example 17: The method of any one of examples 12 through 16, wherein the light source is positioned to emit quasi-coherent light within a range of angles that subtends at least twenty-five percent of the volume of a brain tissue of the patient.

Example 18: The method of example 17, wherein the detector comprises a plurality of detectors positioned in at least one of a first or a second nasal cavity of the patient, a first or a second ear canal of the patient, or any position along a scalp of the patient, wherein each detector of the plurality of detectors is separated from the other detectors by a threshold distance.

Example 19: The method of any one of examples 12 through 18, wherein positioning the light source comprises positioning the light source to emit the quasi-coherent light to a Circle of Willis or a proximal vessel within a head or neck of the patient.

Example 20: The method of any one of examples 1 through 10, wherein determining the physiological characteristic of the patient occurs before the patient is at a hospital.

Example 21: A laser speckle imaging system including: a laser configured to emit at least quasi-coherent light to tissue of a patient, the laser configured to emit the at least quasi-coherent light through a nasal passage of the patient; a detector configured to: detect resulting light passing through at least a portion of the tissue and through an ear canal of the patient; and generate a laser interference pattern image of the tissue, wherein the laser interference pattern image is representative of the portion of the tissue interacting with the at least quasi-coherent light traveling through the nasal passage of the patient; and processing circuitry configured to: receive the laser interference pattern image from the detector; generate a speckle contrast signal or a metric derived from the speckle contrast signal based on the laser interference pattern image; determine, based on the speckle contrast signal or the metric derived from the speckle contrast signal, an occurrence of a stroke; and output a representation of the occurrence of the stroke.

Various examples of the disclosure have been described. Any combination of the described systems, operations, or functions is contemplated. These and other examples are within the scope of the following claims.

What is claimed is:

1. A system comprising:
a light source configured to emit at least quasi-coherent light to tissue of a patient, the light source configured to emit the at least quasi-coherent light through at least one of a nasal passage or an ear canal of the patient;
a detector configured to detect resulting light passing through at least a portion of the tissue and generate an interference pattern image,
wherein the interference pattern image is representative of the portion of the tissue interacting with the quasi-coherent light traveling through at least the nasal passage or the ear canal of the patient, and
processing circuitry configured to:
receive the interference pattern image from the detector;
generate a speckle contrast signal based on the interference pattern image;
determine, based on the speckle contrast signal or a metric derived from the speckle contrast signal, a physiological characteristic of the patient; and
output a representation of the physiological characteristic of the patient.

2. The system of claim 1, wherein the physiological characteristic of the patient comprises one or more flow values of a fluid, one or more flow values within vasculature of the tissue of the patient, an occurrence of an ischemic stroke, or an occurrence of a hemorrhagic stroke.

3. The system of claim 1, wherein the light source is housed in one of a nasoscope or an otoscope.

4. The system of claim 1, wherein the detector comprises a detector optical axis defining a viewing direction of the detector, wherein the light source comprises a source optical axis defining an at least quasi-coherent light emission direction, wherein the detector is positioned such that an angle between the detector optical axis and the source optical axis is greater than 45 degrees and less than 270 degrees.

5. The system of claim 1, further comprising a first housing and a second housing, wherein the light source is disposed within the first housing, wherein the detector is disposed within the second housing different from the first housing, and wherein the first housing and the second housing are configured to be separated from each other by a threshold distance.

6. The system of claim 5, wherein the nasal passage is a first nasal passage, wherein the first housing is a nasoscope configured to position the light source in the first nasal passage of the patient, and wherein the second housing is configured to position the detector in at least one of a second nasal passage of the patient, the ear canal of the patient, or at a scalp of the patient.

7. The system of claim 5, wherein the ear canal is a first ear canal, wherein the first housing is an otoscope configured to position the light source in the first ear canal of the patient, and wherein the second housing is configured to position the detector in at least one of the nasal passage of the patient, a second ear canal of the patient, or at a scalp of the patient.

8. The system of claim 5, wherein the first housing is a head mounted device configured to be position the light source at a first position at a scalp of the patient, wherein the second housing is configured to position the detector in at least one of the nasal passage of the patient, the ear canal of the patient, or at a second position along the scalp of the patient, wherein the interference pattern image is representative of the portion of the tissue interacting with the quasi-coherent light traveling through the scalp of the patient.

9. The system of claim 1, wherein the light source is configured to emit the at least quasi-coherent light with a beam divergence of at least 15 degrees along one axis.

10. The system of claim 9, wherein the detector comprises a plurality of detectors positioned in at least one of the nasal passage of the patient, the ear canal of the patient, or at a scalp of the patient, wherein each detector of the plurality of detectors are separated from each other by a threshold distance.

11. The system of claim 1, wherein light source is configured to be positioned to emit the quasi-coherent light to a Circle of Willis or a proximal vessel within a head or neck of the patient.

12. A method comprising:
positioning a light source to emit at least quasi-coherent light to tissue of a patient through at least one of a nasal passage or an ear canal of the patient;
positioning a detector to detect resulting light passing through at least a portion of the tissue;
generating, by the detector and based on the resulting light, an interference pattern image, wherein the interference pattern image is representative of the portion of the tissue interacting with the quasi-coherent light traveling through at least the nasal passage or the ear canal of the patient;
receiving, by processing circuitry, the interference pattern image from the detector;
generating, by the processing circuitry, a speckle contrast signal based on the interference pattern image;
determining, by the processing circuitry and based on the speckle contrast signal or a metric derived from the speckle contrast signal, a physiological characteristic of the patient; and
outputting a representation of the physiological characteristic of the patient.

13. The method of claim 12, wherein the physiological characteristic of the patient comprises one or more flow values of a fluid, one or more flow values within vasculature of the tissue of the patient, an ischemic stroke, or a hemorrhagic stroke.

14. The method of claim 12, wherein the light source is housed in at least one of a nasoscope or an otoscope.

15. The method of claim 12, wherein the detector comprises a detector optical axis defining a viewing direction of the detector, wherein the light source comprises a source optical axis defining a quasi-coherent light emission direction,
wherein positioning the light source and positioning the detector comprises positioning the light source and positioning the detector such that an angle between detector optical axis and the source optical axis is greater than 45 degrees and less than 270 degrees.

16. The method of claim 12, wherein the light source is disposed within a first housing, wherein the detector is disposed within a second housing different from the first housing, wherein positioning the light source and positioning the detector comprises positioning the first housing separated from the second housing by a threshold distance.

17. The method of claim 12, wherein the light source is positioned to emit quasi-coherent light within a range of angles that subtends at least twenty-five percent of the volume of a brain tissue of the patient.

18. The method of claim 17, wherein the detector comprises a plurality of detectors positioned in at least one of a first or a second nasal cavity of the patient, a first or a second ear canal of the patient, or any position along a scalp of the patient, wherein each detector of the plurality of detectors is separated from the other detectors by a threshold distance.

19. The method of claim 12, wherein positioning the light source comprises positioning the light source to emit the quasi-coherent light to a Circle of Willis or a proximal vessel within a head or neck of the patient.

20. A laser speckle imaging system comprising:
a laser configured to emit at least quasi-coherent light to tissue of a patient, the laser configured to emit the at least quasi-coherent light through a nasal passage of the patient;
a detector configured to:
detect resulting light passing through at least a portion of the tissue and through an ear canal of the patient; and
generate a laser interference pattern image of the tissue, wherein the laser interference pattern image is representative of the portion of the tissue interacting with the at least quasi-coherent light traveling through the nasal passage of the patient; and
processing circuitry configured to:
receive the laser interference pattern image from the detector;
generate a speckle contrast signal or a metric derived from the speckle contrast signal based on the laser interference pattern image;
determine, based on the speckle contrast signal or the metric derived from the speckle contrast signal, an occurrence of a stroke; and
output a representation of the occurrence of the stroke.
